(19) United States
(12) Patent Application Publication
(10) Pub. No.: US 2025/0025391 A1
(43) Pub. Date: Jan. 23, 2025

(54) Sulfate-Free Surfactant System

(71) Applicant: Ethox Chemicals, LLC, Greenville, SC (US)

(72) Inventors: Edward T. Borish, Greenville, SC (US); Nora E. H. Bridenstine, Greenville, SC (US)

(21) Appl. No.: 18/906,224

(22) Filed: Oct. 4, 2024

Related U.S. Application Data
(63) Continuation-in-part of application No. 18/134,900, filed on Apr. 14, 2023.
Application No. 18/134,900 is a continuation-in-part of application No. 17/742,915, filed on May 12, 2022.
Application No. 17/742,915 is a continuation-in-part of application No. 17/468,858, filed on Sep. 8, 2021, now Pat. No. 11,692,107.
Application No. 17/468,858 is a continuation of application No. 17/072,318, filed on Oct. 16, 2020, now Pat. No. 11,491,094.
(60) Provisional application No. 62/923,227, filed on Oct. 18, 2019.

(51) Int. Cl. A61K 8/37; A61K 8/03; A61K 8/34; A61K 8/41

(52) U.S. Cl. CPC A61K 8/375; A61K 8/03; A61K 8/345; A61K 8/41; A61K 2800/10; A61K 2800/30; A61K 2800/48; A61K 2800/596

(57) ABSTRACT

A colloidal dispersion is provide which is particularly suitable for use in a cleaning system or a personal care product. The colloidal dispersion has a Brookfield Yield Value of at least 50 dyn/cm² and the cleaning system comprises a structurant and a sulfate-free surfactant.

### CROSS-REFERENCE TO RELATED APPLICATIONS

The present invention is a continuation-in-part of pending U.S. patent application Ser. No. 18/134,900 filed Apr. 14, 2023 which is, in turn, a continuation-in-part of pending U.S. patent application Ser. No. 17/742,915 filed May 12, 2022 which is, in turn, a continuation-in-part of 1U.S. patent application Ser. No. 17/468,858 filed Sep. 8, 2021 now U.S. Pat. No. 11,692,107 issued Jun. 14, 2023 which is, in turn, a continuation-in-part of U.S. patent application Ser. No. 17/072,318 filed Oct. 16, 2020 now U.S. Pat. No. 11,491,094 issued Nov. 8, 2022 which, in turn, claims priority to expired U.S. Provisional Patent Application No. 62/923,227 filed Oct. 18, 2019 all of which are incorporated herein by reference.

### FIELD OF THE INVENTION

The present invention is related to a surfactant system which functions as a cleaning system or as, or in, a matrix for a variety of materials including for use in a sunscreen or personal care product. More specifically, the present invention is related to a cleaning system comprising a colloidal dispersion, comprising a sulfate-free surfactant and a structurant, which is useful in personal care products and is capable of removing graffiti, grass, dirt, food products, greases, oils, soils, general stains and other materials from a variety of surfaces.

### BACKGROUND

There is an on-going need for improved compositions for cleaners. A particular aspect of this on-going desire is compositions that are water based, water soluble, nonflammable, vegetable-derived, biodegradable, low irritancy, low toxicity, and low volatile organic compounds (VOCs). For example, sulfate-based surfactants are known to be an irritant to mucosal membranes and particularly those membranes associated with the eyes, the skin and the lungs. It has therefore been a societal desire to decrease or remove sulfate-based surfactants from products, particularly liquid products, that can come into contact with the hair and skin of people using the product or in the vicinity of the product being used.

Of particular importance to consumers is viscosity. It is desirable that cleaning products have a viscosity that makes the product easy to control during application and which does not flow to unintended areas. If the viscosity is too low the product spreads quickly and the cleaning agents incorporated therein do not have sufficient time to adequately dislodge the undesirable materials from the surface being cleaned.

With sulfate-based surfactants, and non-aqueous solvent-based systems, viscosity is easily controlled by the addition of salts. In these systems the viscosity generally increases with concentration of salt, within the working range, even up to a viscosity sufficient to form a solid, non-flowable gel. As set forth in U.S. Published Patent Application No. 2009/0257968, particularly suitable salts for increasing viscosity contain cations such as alkali metals, particularly sodium and potassium; and alkaline earth metal salts, such as magnesium and aluminum. However, salts are not effective in controlling the viscosity of non-sulfate aqueous based systems. This has led to efforts to develop a surfactant system suitable for controlling the viscosity of liquid compositions which have a low concentration of sulfate or which are sulfate-free.

Provided herein is a sulfate-free, surfactant system suitable for use with cleaning products, personal care products including sunscreens and the like without limit thereto. The present invention provides a natural-based thickener which is particularly suitable for use in, preferably sulfate-free, cleaning systems, personal care products including sunscreen and the like, and which provides a stable composition with non-Newtonian rheology.

### SUMMARY OF THE INVENTION

The present invention is related to a cleaning system which is optionally and preferably sulfate-free.

More specifically, the present invention is related to a cleaning system comprising a thickener which can provide a non-Newtonian liquid with shear thinning properties.

A particular feature of the invention is the ability to clean material from a surface such as graffiti, grass, dirt, food products, greases, oils, soils, general stains and other materials from a variety of surfaces including hard surfaces and soft surfaces including, but not limited to, natural and synthetic fabric.

A particular advantage of the invention is the compatibility with skin making the surfactant system suitable for use in personal care products.

A particular advantage of the invention is the compatibility with skin making the surfactant system suitable for use in sunscreens.

These and other embodiments, as will be realized, are provided in a cleaning system comprising a colloidal dispersion having a Brookfield Yield Value of at least about 50 dyn/cm² wherein the cleaning system comprises a structurant and a sulfate-free surfactant.

Yet another embodiment is provided in a cleaning system comprising a colloidal dispersion comprising a structurant, a sulfate-free surfactant and a cleaning agent.

Yet another embodiment is provided in a cleaning system comprising a colloidal dispersion comprising a structurant with an HLB of at least about 4 to no more than about 8, a sulfate-free surfactant and a cleaning agent wherein the colloidal dispersion has an HLB of at least about 8.7 to no more than about 10.4 and yield of at least about 50 dyn/cm².

Yet another embodiment is provided in a personal care or personal hygiene product comprising a colloidal dispersion having a Brookfield Yield Value of at least 50 dyn/cm² wherein the colloidal dispersion comprises a structurant and a sulfate-free surfactant.

Yet another embodiment is provided in a colloidal dispersion having a Brookfield Yield Value of at least 50 dyn/cm². The colloidal suspension comprises a structurant, wherein said structurant is a glyceryl ester, and a sulfate-free surfactant.

Yet another embodiment is provided in cleaning system comprising a colloidal dispersion. The colloidal dispersion comprises a structurant, wherein the structurant is a glyceryl ester, a sulfate-free surfactant and a cleaning agent.

### DESCRIPTION

The present invention is related to a cleaning system and more specifically a cleaning system with low sulfate or which is sulfate-free. More specifically, the present invention is related to a cleaning system comprising a colloidal dispersion comprising structurants and sulfate-free surfactants, and optionally cleaning agents, in the form of a stable non-Newtonian liquid.

Of particular interest in the present invention is the ability to remove unwanted surface coatings formed by graffiti, grass, dirt, food products, greases, oils, soils, paints and markers; specifically permanent markers; water-based and organic solvent-based paints, general stains and other materials especially those based on lacquers and acrylics. The instant invention provides a cleaning system comprising a surfactant and a structurant with optional cleaning agents and additional surfactants. For the purposed of the present invention “surfactant” is a sulfate-free surfactant.

The present invention is also suitable for use in personal care products such as soaps, shampoos, body-washes, make-up such as mascara, lipstick and other products comprising a pigment including sunscreens and the like.

The combination of the structurant and surfactant provides viscosity thereby maintaining the cleaning agent in proximity with the surface being cleaned to solubilize, dissolve, weaken or alter the composition of the materials to be removed, or disrupt the bonding of the materials to be removed from the surface, thereby allowing for removal with, preferably, water.

A particular feature of importance for cleaning solutions is stability and particularly stability upon storage. Stability, for the purposes of this invention is the ability of a colloidal dispersion, or non-Newtonian liquid, to remain for extended periods of time, such as at least about 5-6 weeks, without separation. Once a colloidal dispersion has separated it becomes difficult to reestablish by shaking and the like.

Provided herein are structurants which combine with a surfactant to form a stable colloidal dispersion. For the purposes of the present invention the structurant has a hydrophilic-lipophilic balance (HLB) of at least about 4 to no more than about 18. Below an HLB of about 4 and above an HLB of about 18, for the structurant, it is difficult to achieve adequate yield and HLB for the colloidal dispersion to be stable. The colloidal dispersion, which comprises the structurant and sulfate-free surfactant, has an HLB of at least about 8.7 to no more than about 10.8 wherein the colloidal dispersion has a yield of at least about 50 dyn/cm² to a viscosity of no more than about 1,000,000 cps. Below a yield of about 50 dyn/cm² the stability cannot be maintained. Above a viscosity of about 1,000,000 cps the composition becomes difficult to manipulate. The cleaning system comprises the colloidal dispersion and optional cleaning agents and optional solvent. It would be known to those of skill in the art that at high Brookfield Yield Value the Brookfield Viscosity becomes too high to be practical.

The structurant is an ester or fatty acid terminated polyether, fatty alcohol terminated polyether, or fatty amine terminated polyether having an HLB of at least about 4 to no more than about 18, preferably no more than about 12 and even more preferably no more than about 8.

While not wanting to be limited to particular theory, it is hypothesized that the inventive colloidal dispersion forms an unexpected lamellar/spherilitic phase. Surfactants may associate to form various phases intermediate between liquid and crystalline known as liquid crystalline phases. The structure of these phases include cubic, hexagonal cylindrical, lamellar, and multi-lamellar vesicles/spherulites. Formation of these phases is typically accomplished using a combination of ionic surfactants including anionic, cationic and amphoteric surfactants. This differs from the present invention where only nonionic structurants are used in the colloidal dispersion. Shear thinning rheology and the ability to incorporate high levels of oil or water insoluble materials is characteristic of a lamellar/spherilitic phase and consistent with the behavior of the compositions of the present invention and unexpected with nonionic structurants.

The cleaning system, which incorporates the inventive colloidal dispersion, can be applied to a surface by any means common in the art such as by painting with a brush or roller, spraying or applying with a cloth with painting being a particularly suitable method for demonstration of the invention.

A preferred structurant comprises a glyceride, also referred to as glyceride esters or glyceryl esters. The glyceride is formed as the reaction product of glycerin with a molar excess of a mixture of acids. The molar excess provides, on average, primarily mono and diglyceride esters. More specifically, the molar excess is at least about a 10% molar excess to no more than about a 110% molar excess. Even more specifically, for one mole of glycerin at least 1.10 moles of the mixture of acids can be used to no more than about 2.10 moles of the mixture of acids. Below about 1.10 moles of the mixture of acids per mole of glycerin the number of diglyceride esters, based on statistical distribution, is insufficient for the surfactant to provide sufficient thickening efficiency at reasonable concentrations. Above about 2.10 moles of the mixture of acids per mole of glycerin the number of triglyceride esters, based on statistical distribution, increases and is no longer a surfactant suitable for the intended purpose.

The mixture of acids comprises three acids with the first acid having a higher molecular weight than the second acid wherein the second acid has a higher molecular weight than the third acid. The first acid is preferably an acid comprising 16-20 aliphatic carbons and preferably 18 aliphatic carbons. Particularly preferred as the first acid is a methyl branched aliphatic carbon with 16-methylheptadecanoic acid being particularly preferred. The second acid is preferably an alkyl acid of 9-11 carbons which are preferably unbranched and more preferably the second acid is decanoic acid. The third acid is preferably an alkyl acid of 7-9 carbons which are preferably unbranched and more preferably the third acid is octanoic acid.

The mixture of acids comprises, per mole of glycerin, at least about 0.275 to no more than about 0.990 moles of the first acid; at least about 0.250 to no more than about 0.810 moles of the second acid and at least about 0.250 to no more than about 0.810 moles of the third acid. More preferably, the mixture of acids comprises, per mole of glycerin, at least about 0.425 moles of the second acid, even more preferably at least about 0.520 moles of the second acid, even more preferably no more than about 0.580 moles of the second acid. More preferably, the mixture of acids comprises, per mole of glycerin, at least about 0.425 moles of the third acid, even more preferably at least about 0.520 moles of the third acid, even more preferably no more than about 0.580 moles of the third acid.

The reaction of the mixture of acids and glycerin provides a statistical mixture of esters with each ester being the product of a condensation reaction between one randomly selected hydroxyl group on a glycerin molecule and one of either the first acid, second acid or third acid. It is assumed that all acid molecules react and therefore the average number of esters formed per glycerin molecule is approximately the molar ratio of mixed acid to glycerin, which is about 1.1 to 2.1. By way of non-limiting example if 1.5 moles of mixed acid is reacted with 1 mole of glycerin the average number of esters per glycerin molecule is defined as 1.5.

A particularly preferred structurant comprises the reaction of glycerin with isostearic acid, octanoic acid and decanoic acid.

Another preferred structurant comprises glyceryl monostearate.

Another preferred structurant comprises polyglyceryl esters. Polyglyceryl esters are prepared with a sufficient number of fatty acids of sufficient size and polyglycerol with a sufficient number of repeating units to achieve the desired HLB. Preferred polyglyceryl esters are all those described in the INCI Dictionary and Handbook published by the Personal Care Products Council, 1620 L St NW Ste 1200 Washington DC 20036 (www.personalcarecouncil.org).

Particularly preferred polyglyceryl esters are selected from the group consisting of polyglyceryl-2 oleate, polyglyceryl-3 oleate, polyglyceryl-4 oleate, polyglyceryl-6 oleate, polyglyceryl-6 caprylate/caprate polyglyceryl-10 caprylate/caprate, polyglyceryl-10 polyhydroxystearate and polyglyceryl-3 dioleate.

Another preferred structurant comprises polyglyceryl ethers. Polyglyceryl ethers are prepared with a sufficient number of fatty alcohols of sufficient size and polyglycerol with a sufficient number of repeating units to achieve the desired HLB. Preferred polyglyceryl ethers are all those described in the INCI Dictionary and Handbook published by the Personal Care Products Council, 1620 L St NW Ste 1200 Washington DC 20036 (www.personalcarecouncil.org).

The colloidal dispersion comprises at least about 4 wt % structurant to no more than about 60 wt % structurant and at least about 6 wt % surfactant to no more than about 45 wt % surfactant, up to about 90 wt % solvent and an optional cleaning agent. Below about 4 wt % structurant the thickening properties are insufficient and above about 60 wt % structurant an insufficient amount of surfacant is available for optimum performance.

The cleaning system preferably comprises at least about 10 wt % to no more than about 95 wt % colloidal dispersion and about 5 wt % to no more than about 90 wt % of an additional cleaning agent with the balance being solvent. A particularly preferred solvent is water.

Cleaning agents include, without limit thereto: esters, preferably alkyl esters with up to 5 carbons which may be substituted and preferably methyl and ethyl esters, 3-ethoxypropionic ethyl ester, ethyl lactate, soy methyl ester, triglyceride methyl ester, and C₁₋₄ alkyl ester of a C₆₋₂₂ saturated or unsaturated carboxylic acid; propylene carbonate; terpenes, particularly cyclic terpenes and preferably limonene; N-methylpyrrolidone; acetates such as dipropylene glycol methyl ether acetate or dipropylene glycol monomethyl ether acetate, diethylene glycol monoethyl ether acetate; ethyl acetate, n-propyl acetate, n-butyl acetate and isobutyl acetate; ethers such as dipropylene glycol methyl ether or diethylene glycol ethyl ether.

A particularly preferred embodiment comprises a mixture of: about 10-30 wt % structurant; about 20-40 wt % surfactant and about 30-70 wt % cleaning agent with the balance being solvent and other additives as desired.

The structurant and surfactant taken together provide the viscosity and the desired shear thinning rheology properties. It also adds lubricity and emolliency to the finished product thereby allowing the formulator to reduce additional ingredients to achieve preferred aesthetics in the finished product.

The cleaning system can be made by mixing the surfactants with the structurant followed optionally by the addition of the mixture to the solvent, which is preferably water, followed optionally by addition of a cleaning agent. Alternatively all components can be added at the same time, heated and stirred preferably for about 10 minutes. Alternatively, the structurant and optional cleaning agent can be premixed and then added to the surfactant followed optionally by addition of solvent which is preferably water. Another option is to first add the surfactant to the solvent, which is preferably water, followed by addition of the structurant and then the optional cleaning agent or by addition of a premix of the structurant and optional cleaning agent. Depending on the particular composition of preparation method heat may or may not be required. These examples are not meant to be limiting and one of ordinary skill in the art may envision other preparation methods.

A particular feature of the claimed invention is the surprising and unexpected ability to provide a non-Newtonian liquid with shear-thinning properties which provides a satisfactory feel when used by consumers and long term stability. A liquid that has a lower viscosity under high shear than under low shear has shear-thinning rheology. When used in personal hygiene and cleaning products this provides many benefits. The liquid can be dispensed through an orifice, since the viscosity lowers under the higher shear stress of the orifice. However, the viscosity increases once dispensed and therefore the liquid can be applied with minimal flow. Furthermore, the non-Newtonian liquid will suspend bubbles or particulate therein during storage thereby reducing the settling or separation that occurs in the absence of this type of Rheology.

Shear-thinning is quantified by the Brookfield Yield Value (BYV) which is calculated by the equation:

$$BYV = 2 {r}_{1} ( {n}_{1} - {n}_{2} ) / 100$$

wherein n₁ and n₂ are the viscosities at two different spindle speeds, r₁ and r₂, wherein r₂/r₁₌2. BYV is reported as dyn/cm². For the purposes of this invention BYV is measured at ambient temperature, about 25° C. Viscosities were determined at spindle speeds of 10 and 20 rpm using a Brookfield DV-II+ Viscometer and reported as centipoise (cps). A positive BYV indicates shear-thinning.

In some instances the liquid may also be rheopectic as evidenced by a time-dependent shear thickening.

The cleaning system may further comprise a second surfactant, in addition to the surfactant, wherein the second surfactant is at least one surfactant selected from the group consisting of an anionic surfactant, non-ionic surfactant, a cationic surfactant and amphoteric surfactant with the proviso that the HLB of the colloidal dispersion remains within the prestated ranges.

Preferred anionic surfactants include glutamates, taurates, alkanoyl isethionates, alkyl succinates, alkyl sulphosuccinates, N-alkoyl sarcosinates, alkyl phosphates, alkyl ether phosphates, alkyl ether carboxylates, alpha-olefin sulphonates wherein alkyl and acyl groups are preferably 8 to 18 carbons which may be unsaturated. Particularly preferred alpha-olefin sulphonates include the sodium, magnesium, ammonium and mono-, di- and triethanolamine salts thereof. Particularly preferred anionic surfactants include sodium oleyl succinate, ammonium lauryl sulphosuccinate, disodium laureth sulfosuccinate, sodium dodecylbenzene sulphonate, triethanolamine dodecylbenzene sulphonate, sodium cocoyl isethionate, sodium lauroyl isethionate, sodium N-lauryl sarcosinate, sodium lauroyl lactylate, sodium lauroyl glutamate, sodium methyl cocoyl taurate, sodium lauroyl methyl taurate, sodium lauriminodipropionate and mixtures thereof.

Particularly preferred cationic surfactants include quaternized amines, quaternized polysaccharides, alkyl polysaccharides, alkoxylated amines, alkoxylated ether amines, phospholipids, phospholipid derivatives, and mixtures thereof. Quaternized amines comprising three small alkyl groups, such as C₁-C₅ alkyls, and a long change alkyl, such as a C₁₅-C₂₄ alkyl, with behetrimonium chloride are exemplary.

Preferred nonionic surfactants include polyalkylene glycol adducts of alcohols, acids and glycerides; polyglycerin adducts of acids and alcohols, alkyl glucosides, sorbitan esters, fatty acid amides, and polyoxyethylene, polyoxypropylene block copolymers, in particular, the following surfactants, alone or as mixtures. Particularly preferred nonionic surfactants include polyalkylene glycol adducts of alkyl C8-C24 phenols; polyalkylene glycol adducts of C8-C30 alcohols or C8-C30 glycosides, linear or branched, saturated or unsaturated; polyalkylene glycol adducts of C8-C30, linear or branched, saturated or unsaturated fatty acid amides; C8-C30 linear or branched, saturated or unsaturated esters of sorbitol, which are preferably polyalkylene glycol adducts; fatty acid esters of sucrose; C8-C30 alkyl polyglycosides; C8-C30 alkenyl polyglycosides, which are optionally polyalkylene glycol adducts with from 0 to 10 polyalkylene glycol units and comprising from 1 to 15 glucose units; polyalkylene glycol adducts of saturated or unsaturated vegetable oils; derivatives of N-alkyl (C8-C30) glucamine; derivatives of N-acyl C8-C30 methylglucamine; aldobionamides; amine oxides; polyalkylene glycol adducts of silicones also preferred are polyglycerin adducts of fatty acids and alcohols including without limitation adducts with lauric acid or lauryl alcohol containing 1-20 moles of glycerin; polyglycerin adducts of oleic acid or oleyl alcohol containing 1-20 moles of glycerin; polyglycerin adducts of cetearic acid or cetearyl alcohol containing 1-20 moles of glycerin; polyglycerin adducts of octadecic acid or octadecanol containing 1-20 moles of glycerin and alkyl glucosides including without limitation decyl glucoside, lauryl glucoside, coco glucoside, and caprylic/capric glucoside.

Particularly preferred amphoteric surfactants include cocamidopropyl hydroxysultaine, cocamidopropyl betaine and cocobetaine, the sodium salt of diethylaminopropyl laurylaminosuccinamate or mixtures thereof.

The present invention is suitable for use in any application desiring thickening of a sulfate-free surfactant system. The present invention is particularly suitable for cleaning a surface but is also suitable without limitation for use in cleaning surfaces including metal surfaces, porous surfaces, ceramic surfaces, smooth surfaces, painted surfaces, and natural surfaces such as cellulose-based materials.

Without limit thereto, the present invention is particularly useful in applications such as an oven and stove cleaner, a grill and grate cleaner, a degreaser for hard and soft surfaces, a fabric spot remover, a tile and grout cleaner, a bug remover, a tar remover and in a paint stripper, a toilet cleaner, a sealed floor cleaner including wood, laminate, vinyl & tile, and in dish washing. However, the instant invention has been demonstrated to be suitable for use on plastic or urethane-finished wood without damage to the surface.

In use, the cleaning system is applied to a surface to be cleaned. The cleaning system will form a viscous coating on the surface which allows the cleaning agent to interact with the material being cleaned from the surface. After a sufficient amount of time, typically 5 minutes to 10 hours, the cleaning system is removed. The process can be repeated multiple times if desired. Removal of the cleaning system can be accomplished by wiping with a cloth, air impingement, or by flowing solvent, preferably water. The water can be applied by pouring or spraying. In a preferred embodiment the water is applied by spraying under pressure. A particular feature of the invention is that the BYV can be significantly decreased by the addition of water thereby altering the non-Newtonian liquid to a Newtonian liquid which is then more easily removed.

Other additives can be added to the cleaning system to improve aesthetic properties or for additional functionality. Fragrances, colorants, opacifiers, chelating agents, abrasives, anti-deposition agents, brightening agents, UV-absorbers, preservatives, antioxidants, lubricants, penetrants, film formers, detergents, emulsifiers, volatiles, propellants, salts, pH adjusting agents, neutralizing agents, buffers, anti-static agents, absorbents, pigments, dyes, sunscreens and combinations thereof can be employed.

Throughout the specification the term “Cx” refers to x carbons. By way of non-limiting example, C8 refers to a specified group with eight carbons.

For the purposes of the present invention cleaning agents refer to those substances which are intended for use to clean and remove material from a surface, particularly a porous surface, such as concrete, metal or a painted surface.

For the purposes of the present invention the term “sulfate-free” refers to a surfactant system comprising less than 2 wt % sulfate-based surfactant, preferably less than 1 wt % sulfate-based surfactant and most preferably less than a measurable amount of sulfate-based surfactant. A sulfate-based surfactant is one characterized by the following chemical structure:

R—OSO₃⁻¹

where R is a lipophilic group.

Listed ranges for composition are inclusive and include every number with the same number of significant FIGURES in the range. By way of non-limiting example, a stated range of 0.001 to 0.010 would include 0.001, 0.002, 0.003, 0.004, 0.005, 0.006, 0.007, 0.008, 0.009 and 0.010. All concentrations are wt % active. Unless otherwise stated % represents wt %.

In use, contact times can be several minutes to an hour to several hours. One treatment may be sufficient to clean the surface but multiple applications may be necessary for particularly tenacious materials. In general, a preferred method of use is to allow the composition to remain in contact with the soiled substrate and then simply rinse with running water or wipe clean, as for example, with a damp sponge. The composition has the advantage of clinging to vertical surfaces, as for example the walls of an oven. This same property seems to overcome the problem of “wicking” when using solvents in removing stains from cloth.

### EXAMPLES

### Preparation of Colloidal Dispersions

A series of colloidal dispersions were prepared comprising decyl glucoside as the surfactant, 25 wt % soy methyl ester as a cleaning agent, and stucturant as indicated in Table 1 with the balance being water. The BYV, HLB of the structurant and HLB of the colloidal dispersion are reported in Table 1 as is the stability. In Table 1 Ratio is the weight ratio of surfactant to structurant, BYV is the yield in dyn/cm², HLB-ST is the HLB of the structurant, HLB-CD is the HLB of the colloidal dispersion and Stab is the stability with 1 being good stability, 2 being poor stability and 3 being unstable. In Table 1, the colloidal dispersion was considered to have good stability if the colloidal dispersion did not separate at room temperature for about 5-6 weeks. The colloidal dispersion was considered to have poor stability if the colloidal dispersion initially formed a colloidal dispersion but separated at room temperature within less than about 5-6 weeks. A colloidal dispersion was considered to be unstable if the colloidal dispersion remained separated after agitation or separated at room temperature within much less than about 5-6 weeks, ie immediately or within a day or less.

TABLE 1

| | | BYV, | | | |
|---|---|---|---|---|---|
| Structurant | Ratio | dyn/cm² | HLB-ST | HLB-CD | Stab |
| Coco-glycerides | 30/15 | 14,500 | 8 | 10.6 | 1 |
| Glyceryl stearate | 25/12.5 | 10,600 | 4 | 9.3 | 1 |
| Glyceryl | 30/15 | 4,100 | 5 | 9.6 | 1 |
| Monosoyate | | | | | |
| PEG-2 Cocamine | 30/15 | 9,600 | 6 | 10.0 | 1 |
| PEG-2 Oleamine | 30/15 | 8,500 | 4 | 9.6 | 1 |
| PEG-2 Stearamine | 30/15 | 13,900 | 5 | 9.6 | 1 |
| Oleth-2 | 30/15 | 13,700 | 5 | 9.6 | 1 |
| Sorbitan Oleate | 28.13/18.75 | 14,500 | 4 | 8.8 | 1 |
| Coco-glycerides | 30/15 | 500 | 9 | 11.0 | 2 |
| (High C8-C10) | | | | | |
| Sorbitan Oleate | 30/15 | 3,000 | 4 | 9.3 | 2 |
| Caprylic/Capric | 30/15 | 900 | 0 | 8.0 | 2 |
| Triglyceride | | | | | |
| Soybean Oil | 30/15 | 1,000 | 0 | 8.0 | 2 |
| Decyl Oleate | 30/15 | 700 | 0 | 8.0 | 2 |
| Propanediol | 30/15 | 800 | 0 | 8.0 | 2 |
| dicaprylate/caprate | | | | | |
| C12-C15 Alkyl | 30/15 | 800 | 0 | 8.0 | 2 |
| Benzoate | | | | | |
| Cetyl/lauryl/myristyl | 30/15 | 700 | 0 | 8.0 | 2 |
| benzoate | | | | | |
| Glycereth-18 | 30/15 | 300 | 14 | 12.6 | 2 |
| Ethylhexaneoate | | | | | |
| and Glycereth 18 | | | | | |
| PEG-5 Laurate | 30/15 | 100 | 11 | 11.6 | 2 |
| PEG-9 Dioleate | 30/15 | 100 | 8 | 10.6 | 2 |
| Coco-glycerides | 28.13/18.75 | 700 | 9 | 14.7 | 2 |
| (High C8-C10) | | | | | |
| Coco-glycerides | 31.25/12.5 | 500 | 9 | 12.1 | 2 |
| (High C8-C10) | | | | | |
| Soybean Oil | 28.13/18.75 | 1,000 | 0 | 7.2 | 2 |
| Soybean Oil | 31.25/12.5 | 1,000 | 0 | 8.5 | 2 |
| Oleth-5 | 30/15 | 4,800 | 9 | 11 | 2 |
| Soybean Oil | 25/25 | 4,200 | 0 | 6 | 2 |
| Sorbitan Oleate | 31.25/12.5 | 700 | 4 | 9.8 | 2 |
| PEG-5 Cocamine | 30/15 | 0 | 10.5 | 11.5 | 3 |
| PEG-5 Oleate | 30/15 | 0 | 9 | 11 | 3 |
| PEG-5 Cocamine | 34.4/17.2 | 0 | 10.5 | 11.5 | 3 |

### Preparation of Glycerides

Ester 1 would be prepared by reacting 1 mole of glycerin (99.7%) with 2 moles of a mixture of acids where the acid mix comprises 25 wt % isostearic acid and 34 wt % octanoic acid and 41 wt % decanoic acid. The glyceride is formed using any method by those of ordinary skill in the art or any means common in the art such as transesterification and direct esterification.

Ester 2 would be prepared by the same procedure as Ester 1 using slightly more than 1 mole of a mixture of acids where the acid mix is 36 wt % isostearic acid and 32 wt % octanoic acid and 32 wt % decanoic acid.

Ester 3 would be prepared by the same procedure as Ester 2 with 26 wt % isostearic acid, 22 wt % octanoic acid and 22 wt % decanoic acid per mole of glycerin.

### Preparation of Mixtures

### Example 1

25 grams of limonene or soy methyl ester was mixed with 15 grams of Ester 3 and heated to about 50-60° C. with mixing for several minutes. The mixture was then added to an aqueous solution of decyl glucoside (50% active) with mixing resulting in a thick cream having a composition which is decyl glucoside 30% active, Ester 3 15%, soy methyl ester or limonene 25%, and water 30%.

### Example 2

17.5 grams of Ester 3 was added to 62.5 grams of an aqueous solution of decyl glucoside (50% active) at room temperature. To this mixture was added 20 grams of limonene or soy methyl ester with mixing at room temperature resulting in a thickened mixture. Both example mixtures were tested as follows and found be effective in removing graffiti. Apply (spread, brush, roll) product to surface to be treated, for example subway tile. After 30 minutes brush lightly then reapply more product. After 30 minutes brush lightly again then rinse with running water, preferably using a power washer along with additional brushing if necessary. Finally, rinse clean.

### Example 3. Oven and Stove Cleaner

The mixture of Example 1 made using soy methyl ester was placed on the soiled porcelain enamel surface of an electric oven. After about 1 hour at room temperature a green plastic scrub pad was used to gently scrub the surface for several seconds. The area was then simply wiped clean using a moist kitchen sponge. The soil could not be removed by scrubbing and rinsing in the absence of the oven and stove cleaner.

### Example 4. Cleanser for Removing Baked-on/Polymerized Grease from Tile

The mixture of Example 3 was applied to the vertical tile backsplash of a kitchen stove coated with baked-on/polymerized grease that could not be removed using convention hard surface cleaner. After about an hour the treated surface was wiped clean with a damp kitchen sponge.

### Example 5. Cleanser for Removing Paint from Fabric

The mixture of Example 3 was applied using cotton swabs to cotton fabric with paint that was dried on for over a year. After about 10 minutes the fabric was rinsed briefly under running and the paint was found to be completely removed.

### Example 6. Water-Proof Mascara

A portion of fabric (94% polyester & 6% spandex) about 1 in² had mascara, L'Oreal Voluminous Lash Paradise, black water proof applied to it. Then mixture of Example 3 was applied. After about an hour the fabric was rinsed under running water with gentle rubbing by hand. Most but not of the mascara was removed. However, after a second application as above the mascara was completely removed.

### Example 7. Lipstick

A portion of the same fabric as above in Example 3 was treated with lipstick with the same results as above.

### Example 8. Grass and Dirt

Cotton fabric with ground in grass and dirt stains was treated as in Example 3 with the same results as above.

### Example 9. Blood

Samples of both cotton and polyester fabric stained with blood were treated with the mixture of Example 3. After about an hour the cotton fabric was rinsed under running water and followed by gentle rubbing by hand until most of the stain was removed. A second application resulted in the stain being almost completely removed. After about an hour the stain on polyester was completely removed by rinsing under running water with gentle rubbing by hand.

### Example 10. Mustard

Samples of both cotton and polyester fabric stained with mustard were treated with the mixture of Example 3 with the same results as above.

### Example 11. Cleanser for Bicycle Dirt & Grease

Samples of both cotton and polyester fabric stained with dirty bicycle chain grease were treated with the mixture of Example 3. After about 1 hour the stain was found to be completely removed after rinsing under water with gentle rubbing by hand.

### Example 12. Polyurethane

A 4 in² section of glossy floorboard prefinished with polyurethane was treated with the mixture of Example 3. After about an hour the treated area was scrubbed using a plastic brush and rinsed under running water with rubbing by hand. The treated area appeared to be unaffected by the treatment and appeared as glossy as before treatment. A second treatment was applied except the contact time was increased to about 24 hours. Again, the treated area appeared to be unaffected by the treatment and appeared as glossy as before treatment.

### Example 13. Cleanser for Silicone-Based Chairlift Grease

A nylon ski jacket heavily soiled with silicone-based chairlift grease was treated with a liberal amount of the mixture of Example 3. After about 2 hours the stain was completely removed after rinsing with water.

### Example 14. Cleanser for Cooking Oil Stain

A shirt (92% polyester & 8% spandex) stained with olive oil was treated with the mixture of Example 3. After 10 minutes it was rinsed and the stain was completely removed.

### Example 15. Cleanser for Facial Makeup

A shirt collar stained with Clinique liquid foundation was treated with the mixture of Example 3. After about 15 minutes it was rinsed and the stain was completely removed.

### Example 16. Wine Stain Remover

Red wine was applied to a dish towel was treated by immediate application of the mixture of Example 3. After about 10 minutes it was rinsed and the wine was completely removed.

### Cleaning-Agent-Free Examples

A series of formulations were prepared in water as the only solvent, as indicated in Table 2 where % Surfactant and % Structurant are percent by weight on an active/solids basis. The surfactant, structurant and water were mixed and the colloidal dispersion was formed as described herein.

Sample 1 was an opaque white, orange-pink tinted thin liquid that separated in 1 day. The sample was prepared by heating between 40-50° C. and stirring for about 10 min. followed by cooling to room temperature (RT). Water was added with stirring at RT. Sample 1 is a diluted example of Sample 2 which demonstrates the ability to disrupt the non-Newtonian properties by the addition of water for removal.

Sample 2 was an opaque white, orange-pink tinted thick cream. The sample was prepared by heating between 40-50° C., stirring for 10 min. and then cooling to RT.

Sample 3 was an opaque off-white, yellow tinted thick cream. The sample was prepared by heating between 40-50° C. and stirring 10 min. before cooling to RT.

Sample 4 was an opaque off-white creamy thin liquid. Sample 4 was prepared by adding water to sample 3 with stirring at RT. Sample 4 and Sample 1 demonstrate the ability to decrease the viscosity partially is necessary for the purposes of the application.

Sample 5 was an opaque off-white creamy thin liquid. The sample was heated between 40-50° C., stirred 10 min., then cooled to RT.

Sample 6 was an opaque white thin liquid prepared by adding water to Sample 5 with stirring at RT. Sample 6 separated in 14 days.

Sample 7 was an opaque white creamy fluid liquid that was stable for 41 days. Sample 7 was prepared by heating at between 40-50° C., stirring for 10 min. then cooling to RT. Water was added with stirring at RT.

Sample 8 was an opaque white very thick gel prepared by heating between 40-50° C. and stirring for 10 min. followed by cooling to RT.

Sample 9 was an opaque white creamy pourable liquid prepared by heated between 40-50° C. and stirring for 10 min. and cooling to RT. Water was added with stirring at RT.

Sample 10 was an opaque white creamy gel prepared by heating between 40-50° C., stirring for 10 min. and cooling to RT.

Sample 11 was an opaque white, semi-solid, non-fluid gel prepared by heating between 40-50° C., stirring for 10 min. the cooling to RT.

Sample 12 was an opaque white fluid cream prepared by adding water to Sample 11 with stirring at RT. Sample 12 was stable for 36 days.

Sample 13 was an opaque white, semi-solid, non-fluid gel formed by heating between 40-50° C. with stirring for 10 min. then cooling to RT.

Sample 14 was an opaque white liquid rich frothy texture formed by adding water to Sample 13 with stirring at RT. Sample 14 was stable for 3 hours.

Sample 15 was an opaque off-white semi-solid non-fluid cream formed by heating between 40-50° C. and stirring for 10 min. followed by cooling to RT.

Sample 16 was an opaque, white thin, fluid liquid prepared by adding water to Sample 15 with stirring at RT.

Sample 17 was an opaque light orange pink tinted semi-solid, non-fluid cream formed by heating between 40-50° C. and stirring for 10 min. then cooling to RT.

Sample 18 was an opaque white pink tinted fluid cream formed by adding water to Sample 17 and heating to between 50-70° C. with stirring for 10 min. followed by cooling to RT. Sample 18 was stable for 11 days.

Sample 19 was an opaque white semi-solid cream formed by heating to between 40-50° C. and stirring for 10 min. followed by cooling to RT.

Sample 20 was an opaque white fluid cream formed by adding water to Sample 19 with heating between 50-70° C. with stirring for 10 min. followed by cooling to RT.

Sample 21 was an opaque white aerated semi-solid non-fluid gel formed by heating to between 40-50° C. and stirring for 10 min. followed by cooling to RT.

Sample 22 was an opaque white frothy, thin liquid formed by adding water to Sample 21 at between 50-70° C. with stirring for 10 min. then cooling to RT. Sample 22 was stable for 19 days.

Sample 23 was an opaque off-white semi-solid gel formed by heating to between 40-50° C., stirring for 10 min. then cooling to RT.

Sample 24 was an opaque white thin liquid formed by adding water to sample 23 at between 50-70 C with stirring for 10 min. then cooling to RT. Sample 24 was not non-Newtonian.

Sample 25 was an opaque white clay like solid formed by heating to between 40-50° C. and stirring 10 min. then cooling to RT.

Sample 26 was an opaque white cream formed by adding water to Sample 25 between 50-70° C. with stirring for 10 min. then cooling to RT. Sample 26 was stable for 15 days.

Sample 27 was an opaque white thick cream formed by heating to between 40-50° C., stirring for 10 min. then cooling to RT.

Sample 28 was an opaque white creamy pourable liquid formed by adding water to Sample 27 and heating to between 50-70° C. with stirring for 10 min. then cooled to RT.

Sample 29 was an opaque white semi-solid non-fluid cream formed by mixing at RT with stirring for 1-2 min. until homogenous.

Sample 30 was an opaque white fluid cream formed by adding water to Sample 29 with stirring at RT for 2 min.

Sample 31 was an opaque white fluid cream formed by mixing at RT with stirring for 1-2 min. until homogenous.

Sample 32 was an opaque white fluid cream formed by adding water to Sample 31 with stirring at RT for 2 min.

Sample 33 was an opaque off-white semi-solid, non-fluid gel formed by mixing at RT with stirring for 1-2 min. until homogenous.

Sample 34 was an opaque off-white semi-solid, non-fluid gel formed by adding water to Sample 33 with stirring at RT for 2 min.

Sample 35 was an opaque off-white semi-solid, non-fluid gel formed by mixing at RT with stirring for 1-2 min. until homogenous.

Sample 36 was an opaque off-white fluid whipped cream formed by adding water to Sample 35 with stirring at RT for 2 min.

Sample 37 was an opaque white semi-solid non-fluid gel formed by mixing at RT then heating to 31° C. for 1-2 min with stirring to melt and incorporate structurant until homogenous.

Sample 38 was an opaque white semi-solid non-fluid gel formed by adding water to Sample 37 and stirring at RT for 2 min. Sample 38 was stable for 4 days.

Sample 39 was an opaque white somewhat fluid when tapped cream formed by mixing at RT and heating to 310 C for 1-2 min. with stirring to melt and incorporate structurant until homogenous.

Sample 40 was an opaque white creamy liquid formed by adding water to Sample 29 with stirring at RT for 2 min.

Sample 41 was an opaque white semi-solid, non-fluid gel formed by dissolving the amine in water at 80-90° C., cooling to 60-70° C., adding structurant with mixing, then cooling to RT.

Sample 42 was an opaque white semi-solid, non-fluid gel formed by dissolving the amine in water at 80-90° C., cooling to 60-70° C., adding structurant with mixing, then cooling to RT.

Sample 43 was an opaque white semi-solid, non-fluid gel formed by dissolving the amine in water at 80-90° C., cooling to 60-70° C., adding structurant with mixing, then cooling to RT.

Sample 44 was an opaque white semi-solid, non-fluid gel formed by dissolving the amine in water at 80-90° C., cooling to 60-70° C., adding structurant with mixing, then cooling to RT.

Sample 45 was an opaque white very thick, semi-solid, non-fluid gel formed by dissolving the amine in water at 80-90° C., cooling to 60-70° C., adding structurant with mixing, then cooling to RT.

Sample 46 was an opaque white semi-solid, non-fluid gel formed by dissolving the amine in water at 80-90° C., cooling to 60-70° C., adding the structurant with mixing, then cooling to RT.

Sample 47 was an opaque orange-white aerated semi-solid, non-fluid gel formed by heating to between 40-50° C. with stirring for 10 min. then cooling to RT.

Sample 48 was an opaque orange-white aerated semi-solid, non-fluid cream formed by adding water to Sample 47 with stirring at RT.

Sample 49 was an opaque off-white aerated semi-solid, non-fluid gel formed by heating at between 40-50° C. with stirring for 10 min. then cooling to RT.

Sample 50 was an opaque off-white aerated semi-solid, non-fluid cream formed by adding water to Sample 49 with stirring at RT.

Sample 51 was an opaque off-white aerated semi-solid, non-fluid gel formed by heating between 40-50° C. with stirring for 10 min. then cooling to RT.

Sample 52 was an opaque off-white aerated semi-solid, non-fluid cream formed by adding water to Sample 51 with stirring at RT.

Sample 53 was an opaque off-white aerated semi-solid, somewhat fluid gel formed by heating between 40-50° C. with stirring for 10 min. then cooling to RT.

Sample 54 was an opaque off-white aerated semi-solid, somewhat fluid cream formed by adding water to Sample 53 with stirring at RT.

Sample 55 was an opaque off-white aerated semi-solid, non-fluid gel formed by heating between 40-50° C. and stirring for 10 min. The sample was then heated at 60-65° C. for 5 min with stirring to melt and incorporate the structurant followed by cooling to RT.

Sample 56 was an opaque off-white aerated semi-solid, somewhat fluid cream formed by adding water to Sample 55 with stirring at RT. Sample 56 was stable for 1 day.

Sample 57 was an opaque off-white aerated semi-solid, non-fluid gel formed by heating between 40-50° C. with stirring for 10 min. then cooling to RT.

Sample 58 was an opaque off-white aerated semi-solid, non-fluid cream formed by adding water to Sample 57 with stirring at RT.

Sample 59 was an opaque off-white creamy pourable liquid formed by mixing at RT and stirring with a stir rod for 1-2 min.

Sample 60 was an opaque white liquid formed by adding water to Sample 59 at RT for 1-2 mixed with stir rod. Sample 60 was stable for 7 days.

Sample 61 was a clear aerated fluid gel formed by mixing at RT and stirring with stir rod for 1-2 min.

Sample 62 was an opaque white fluid prepared by adding water to Sample 61 at RT for 1-2 min. while mixing with a stir rod.

Sample 63 was an opaque, white semi-solid non-fluid gel prepared by mixing at RT and stirring with a stir rod for 1-2 min.

Sample 64 was an opaque, off-white semi-solid non-fluid gel prepared by mixing water to Sample 63 at RT for 1-2 min. while mixing with a stir rod.

Sample 65 was an opaque, off-white semi-solid non-fluid gel formed by mixing at RT and stirring with a stir rod for 1-2 min.

Sample 66 was an opaque, off-white semi-solid non-fluid gel prepared by adding water to Sample 65 at RT and mixing for 1-2 min. with a stir rod.

Sample 67 was an opaque white non-fluid, thick semi-solid prepared by mixing at 45-55° C. for 10 min, stirring with a stir rod, and cooling to RT.

Sample 68 was an opaque white non-fluid soft semi-solid prepared by adding water to Sample 67 at RT for 1-2 min. while mixing with a stir rod. Sample 68 was stable for 1 day.

Sample 69 was an opaque, off-white semi-solid non-fluid gel formed by mixing at 45° C. for 10 min, stirring with a stir rod and cooling to RT.

Sample 70 was an opaque, off-white semi-solid non-fluid gel formed by adding water to Sample 69 at RT while mixing for 1-2 min. with a stir rod.

TABLE 2

| | | | % | | |
|---|---|---|---|---|---|
| | | | Structurant/ | Brookfield | BYV, |
| Sam- | | | % | Viscosity cps, | dyn/ |
| ple | Surfactant | Structurant | Surfactant | spindle, rpm | cm² |
| 1 | Cocamido | Sorbitan | 16.67/10.00 | 120 TC@10 | 20 |
| | propyl | Oleate | | 20 TC@20 | |
| | Betaine | | | | |
| 2 | Cocamido | Sorbitan | 33.33/20.00 | 65000 TF@10 | 6400 |
| | propyl | Oleate | | 33000 TF@20 | |
| | Betaine | | | | |
| 3 | Cocamido | Sorbitan | 39.8/18.06 | 112000 TF@10 | 14000 |
| | propyl | Oleate | | 42000 TF@20 | |
| | Betaine | | | | |
| 4 | Cocamido | Sorbitan | 19.90/9.03 | 7000 TF@10 | 700 |
| | propyl | Oleate | | 3500 TF@20 | |
| | Betaine | | | | |
| 5 | Cocamido | Sorbitan | 26.50/22.05 | 7000 TF@10 | 700 |
| | propyl | Oleate | | 3500 TF@20 | |
| | Betaine | | | | |
| 6 | Cocamido | Sorbitan | 13.25/11.03 | 1000 TF@10 | 200 |
| | propyl | Oleate | | 0 TF@20 | |
| | Betaine | | | | |
| 7 | Cocamido | Oleth-2 | 16.67/10.00 | 10000 TF@10 | 400 |
| | propyl | | | 8000 TF@20 | |
| | Betaine | | | | |
| 8 | Cocamido | Oleth-2 | 33.33/20.00 | 80000 TF@10 | 3800 |
| | propyl | | | 61000 TF@20 | |
| | Betaine | | | | |
| 9 | Cocamido | Glyceryl | 16.67/10 | 16000 TF@10 | 1300 |
| | propyl | Monosoyate | | 9500 TF@20 | |
| | Betaine | | | | |
| 10 | Cocamido | Glyceryl | 33.33/20.00 | 32000 TF@10 | 2200 |
| | propyl | Monosoyate | | 21000 TF@20 | |
| | Betaine | | | | |
| 11 | Cocamido | Coco- | 33.33/20.00 | 369000 TF@10 | 33400 |
| | propyl | glycerides | | 202000 TF@ 20 | |
| | Betaine | | | | |
| 12 | Cocamido | Coco- | 16.67/10.00 | 7000 TF@10 | 600 |
| | propyl | glycerides | | 4000 TF@20 | |
| | Betaine | | | | |
| 13 | Cocamido | PEG-2 | 33.33/20.00 | 700000 TF@10 | 54800 |
| | propyl | Stearamine | | 426000 TF@20 | |
| | Betaine | | | | |
| 14 | Cocamido | PEG-2 | 16.67/10.00 | 8000 TF@10 | 1100 |
| | propyl | Stearamine | | 2500 TF@20 | |
| | Betaine | | | | |
| 15 | Cocamido | Glyceryl | 33.33/20.00 | 117000 TF@10 | 8700 |
| | propyl | Oleate (and) | | 73500 TF@20 | |
| | Betaine | Caprylic/ | | | |
| | | Capric | | | |
| | | Glycerides | | | |
| 16 | Cocamido | Glyceryl | 16.67/10.00 | 7000 TF@10 | 500 |
| | propyl | Oleate (and) | | 4500 TF@20 | |
| | Betaine | Caprylic/ | | | |
| | | Capric | | | |
| | | Glycerides | | | |
| 17 | Sodium | Sorbitan | 33.33/20.00 | 53000 TF@10 | 4100 |
| | Lauroyl | Oleate | | 32500 TF@20 | |
| | Sarcosinate | | | | |
| 18 | Sodium | Sorbitan | 20.00/12.00 | 1000 TF@10 | 100 |
| | Lauroyl | Oleate | | 500 TF@20 | |
| | Sarcosinate | | | | |
| 19 | Sodium | Glyceryl | 33.33/20.00 | 261000 TF@10 | 22400 |
| | Lauroyl | Monosoyate | | 149000 TF@20 | |
| | Sarcosinate | | | | |
| 20 | Sodium | Glyceryl | 20.00/12.00 | 6000 TF@10 | 600 |
| | Lauroyl | Monosoyate | | 3000 TF@20 | |
| | Sarcosinate | | | | |
| 21 | Sodium | Oleth-2 | 33.33/20.00 | 28000 TF@10 | 1900 |
| | Lauroyl | | | 18500 TF@20 | |
| | Sarcosinate | | | | |
| 22 | Sodium | Oleth-2 | 20.00/12.00 | 3000 TF@10 | 200 |
| | Lauroyl | | | 2000 TF@20 | |
| | Sarcosinate | | | | |
| 23 | Sodium | Glyceryl | 33.33/20.00 | 139000 TF@10 | 11600 |
| | Lauroyl | Oleate (and) | | 81000 TF@20 | |
| | Sarcosinate | Caprylic/ | | | |
| | | Capric | | | |
| | | Glycerides | | | |
| 24 | Sodium | Glyceryl | 20.00/12.00 | 1000 TF@10 | 0 |
| | Lauroyl | Oleate (and) | | 1000 TF@20 | |
| | Sarcosinate | Caprylic/ | | | |
| | | Capric | | | |
| | | Glycerides | | | |
| 25 | Sodium | PEG-2 | 33.33/20.00 | not measurable | |
| | Lauroyl | Stearamine | | solid | |
| | Sarcosinate | | | | |
| 26 | Sodium | PEG-2 | 20.00/12.00 | 23000 TF@10 | 2100 |
| | Lauroyl | Stearamine | | 12500 TF@20 | |
| | Sarcosinate | | | | |
| 27 | Sodium | Coco- | 33.33/20.00 | 134000 TF@10 | 10200 |
| | Lauroyl | glycerides | | 83000 TF@20 | |
| | Sarcosinate | | | | |
| 28 | Sodium | Coco- | 20.00/12.00 | 4000 TF@10 | 300 |
| | Lauroyl | glycerides | | 2500 TF@20 | |
| | Sarcosinate | | | | |
| 29 | Disodium | Sorbitan | 23.00/24.64 | 19000 TF@10 | 1600 |
| | Laureth | Oleate | | 11000 TF@20 | |
| | Sulfo- | | | | |
| | succinate | | | | |
| 30 | Disodium | Sorbitan | 20.00/21.44 | 5000 TF@10 | 100 |
| | Laureth | Oleate | | 4500 TF@20 | |
| | Sulfo- | | | | |
| | succinate | | | | |
| 31 | Disodium | Glyceryl | 23.00/24.64 | 22000 TF@10 | 1900 |
| | Laureth | Monosoyate | | 12500 TF@20 | |
| | Sulfo- | | | | |
| | succinate | | | | |
| 32 | Disodium | Glyceryl | 20.00/21.44 | 9000 TF@10 | 800 |
| | Laureth | Monosoyate | | 5000 TF@20 | |
| | Sulfo- | | | | |
| | succinate | | | | |
| 33 | Disodium | Oleth-2 | 23.00/24.64 | 164000 TF@10 | 8200 |
| | Laureth | | | 123000 TF@20 | |
| | Sulfo- | | | | |
| | succinate | | | | |
| 34 | Disodium | Oleth-2 | 20.00/21.44 | 135000 TF@10 | 12900 |
| | Laureth | | | 70500 TF@20 | |
| | Sulfo- | | | | |
| | succinate | | | | |
| 35 | Disodium | Glyceryl | 23.00/24.64 | 80000 TF@10 | 7200 |
| | Laureth | Oleate (and) | | 44000 TF@20 | |
| | Sulfo- | Caprylic/ | | | |
| | succinate | Capric | | | |
| | | Glycerides | | | |
| 36 | Disodium | Glyceryl | 20.00/21.44 | 83000 TF@10 | 7000 |
| | Laureth | Oleate (and) | | 48000 TF@20 | |
| | Sulfo- | Caprylic/ | | | |
| | succinate | Capric | | | |
| | | Glycerides | | | |
| 37 | Disodium | PEG-2 | 23.00/24.64 | 349000 TF@10 | 30800 |
| | Laureth | Stearamine | | 195000 TF@20 | |
| | Sulfo- | | | | |
| | succinate | | | | |
| 38 | Disodium | PEG-2 | 20.00/21.44 | 57000 TF@10 | 4700 |
| | Laureth | Stearamine | | 33500 TF@20 | |
| | Sulfo- | | | | |
| | succinate | | | | |
| 39 | Disodium | Coco- | 23.00/24.64 | 23000 TF@10 | 2000 |
| | Laureth | glycerides | | 13000 TF@20 | |
| | Sulfo- | | | | |
| | succinate | | | | |
| 40 | Disodium | Coco- | 20.00/21.44 | 7000 TF@10 | 900 |
| | Laureth | glycerides | | 2500 TF@20 | |
| | Sulfo- | | | | |
| | succinate | | | | |
| 41 | Behetri- | Sorbitan | 14.00/13.94 | 434000 TF@10 | 37800 |
| | monium | Oleate | | 245000 TF@20 | |
| | Chloride | | | | |
| 42 | Behetri- | Glyceryl | 14.00/13.94 | 129000 TF@10 | 10600 |
| | monium | Monosoyate | | 76000 TF@20 | |
| | Chloride | | | | |
| 43 | Behetri- | Oleth-2 | 14.00/13.94 | 275000 TF@10 | 22800 |
| | monium | | | 161000 TF@20 | |
| | Chloride | | | | |
| 44 | Behetri- | Glyceryl | 14.00/13.94 | 193000 TF@10 | 14400 |
| | monium | Oleate (and) | | 121000 TF@20 | |
| | Chloride | Caprylic/ | | | |
| | | Capric | | | |
| | | Glycerides | | | |
| 45 | Behetri- | PEG-2 | 14.00/13.94 | 794000 TF@10 | 62200 |
| | monium | Stearamine | | 483000 TF@20 | |
| | Chloride | | | | |
| 46 | Behetri- | Coco- | 14.00/13.94 | 260000 TF@10 | 21200 |
| | monium | glycerides | | 154000 TF@20 | |
| | Chloride | | | | |
| 47 | Decyl | Sorbitan | 33.30/36.02 | 205000 TF@10 | 17000 |
| | Glucoside | Oleate | | 120000 TF@20 | |
| | and Sodium | | | | |
| | Lauroyl | | | | |
| | Lactylate | | | | |
| 48 | Decyl | Sorbitan | 20.00/21.60 | 52000 TF@10 | 4700 |
| | Glucoside | Oleate | | 28500 TF@20 | |
| | and Sodium | | | | |
| | Lauroyl | | | | |
| | Lactylate | | | | |
| 49 | Decyl | Oleth-2 | 33.30/36.02 | 160000 TF@10 | 14800 |
| | Glucoside | | | 86000 TF@20 | |
| | and Sodium | | | | |
| | Lauroyl | | | | |
| | Lactylate | | | | |
| 50 | Decyl | Oleth-2 | 20.00/21.60 | 85000 TF@10 | 7600 |
| | Glucoside | | | 47000 TF@20 | |
| | and Sodium | | | | |
| | Lauroyl | | | | |
| | Lactylate | | | | |
| 51 | Decyl | Glyceryl | 33.30/36.02 | 31000 TF@10 | 2400 |
| | Glucoside | Oleate (and) | | 19000 TF@20 | |
| | and Sodium | Caprylic/ | | | |
| | Lauroyl | Capric | | | |
| | Lactylate | Glycerides | | | |
| 52 | Decyl | Glyceryl | 20.00/21.60 | 30000 TF@10 | 2300 |
| | Glucoside | Oleate (and) | | 18500 TF@20 | |
| | and Sodium | Caprylic/ | | | |
| | Lauroyl | Capric | | | |
| | Lactylate | Glycerides | | | |
| 53 | Decyl | Glyceryl | 33.30/36.02 | 60000 TF@10 | 5200 |
| | Glucoside | Monosoyate | | 34000 TF@20 | |
| | and Sodium | | | | |
| | Lauroyl | | | | |
| | Lactylate | | | | |
| 54 | Decyl | Glyceryl | 20.00/21.60 | 51000 TF@10 | 4400 |
| | Glucoside | Monosoyate | | 29000 TF@20 | |
| | and Sodium | | | | |
| | Lauroyl | | | | |
| | Lactylate | | | | |
| 55 | Decyl | PEG-2 | 33.30/36.02 | 221000 TF@10 | 17800 |
| | Glucoside | Stearamine | | 132000 TF@20 | |
| | and Sodium | | | | |
| | Lauroyl | | | | |
| | Lactylate | | | | |
| 56 | Decyl | PEG-2 | 20.00/21.60 | 49000 TF@10 | 2200 |
| | Glucoside | Stearamine | | 38000 TF@20 | |
| | and Sodium | | | | |
| | Lauroyl | | | | |
| | Lactylate | | | | |
| 57 | Decyl | Coco- | 33.30/36.02 | 112000 TF@10 | 4800 |
| | Glucoside | glycerides | | 88000 TF@20 | |
| | and Sodium | | | | |
| | Lauroyl | | | | |
| | Lactylate | | | | |
| 58 | Decyl | Coco- | 20.00/21.60 | 57000 TF@10 | 4800 |
| | Glucoside | glycerides | | 33000 TF@20 | |
| | and Sodium | | | | |
| | Lauroyl | | | | |
| | Lactylate | | | | |
| 59 | Decyl | Sorbitan | 26.00/37.00 | 28000 TF@10 | 2000 |
| | Glucoside | Oleate | | 18000 TF@20 | |
| 60 | Decyl | Sorbitan | 17.50/25.00 | 3000 TF@10 | 200 |
| | Glucoside | Oleate | | 2000 TF@20 | |
| 61 | Decyl | Oleth-2 | 26.00/37.00 | 12000 TF@10 | 1000 |
| | Glucoside | | | 7000 TF@20 | |
| 62 | Decyl | Oleth-2 | 17.50/25.00 | 11000 TF@10 | 800 |
| | Glucoside | | | 7000 TF@20 | |
| 63 | Decyl | Glyceryl | 26.00/37.00 | 165000 TF@10 | 12800 |
| | Glucoside | Oleate (and) | | 101000 TF@20 | |
| | | Caprylic/ | | | |
| | | Capric | | | |
| | | Glycerides | | | |
| 64 | Decyl | Glyceryl | 17.50/25.00 | 40000 TF@10 | 3500 |
| | Glucoside | Oleate (and) | | 22500 TF@20 | |
| | | Caprylic/ | | | |
| | | Capric | | | |
| | | Glycerides | | | |
| 65 | Decyl | Glyceryl | 26.00/37.00 | 115000 TF@10 | 8400 |
| | Glucoside | Monosoyate | | 73000 TF@20 | |
| 66 | Decyl | Glyceryl | 17.50/25.00 | 18000 TF@10 | 1200 |
| | Glucoside | Monosoyate | | 12000 TF@20 | |
| 67 | Decyl | PEG-2 | 26.00/37.00 | not measureable | |
| | Glucoside | Stearamine | | solid | |
| 68 | Decyl | PEG-2 | 17.50/25.00 | 48000 TF@10 | 4600 |
| | Glucoside | Stearamine | | 25000 TF@20 | |
| 69 | Decyl | Coco- | 26.00/37.00 | 217000 TF@10 | 18000 |
| | Glucoside | glycerides | | 127000 TF@20 | |
| 70 | Decyl | Coco- | 17.50/25.00 | 47000 TF@10 | |
| | Glucoside | glycerides | | 26500 TF@20 | 4100 |

### Sunscreen Examples

A series formulations were prepared comprising decyl glucoside as the surfactant, a structurant, a sunscreen, optionally a non-aqueous solvent as indicated in Table 3 with the balance being water. The formulations were prepared by combining the structurant, sunscreen and non-aqueous solvent (if used) and mixing until uniform. The mixture was heated to about between 40-50 C for about 10 minutes with mixing. The surfactant was added to a separate vessel without heating. Then, with adequate mixing, the mixture was added to the surfactant and mixed for about 5 minutes. The viscosity and BYV for the series of formulations is reported in Table 3. These results indicate that the formulations are all pseudo plastic and all have a BYV sufficient to be stable. The Sun Protection Factor (SPF) of the formulation of Example 71 and 71a were determined to be >40 consistent with them being highly effective sunscreen formulations.

TABLE 3

| | | | | Non-Aqueous | Brookfield | |
|---|---|---|---|---|---|---|
| | Surfactant | Structurant | Sunscreen | Solvent | Viscosity cps, | BYV |
| Sample | % | % | % | % | spindle, rpm | dyn/cm² |
| 71 | Decyl | Glyceryl | Zinc Oxide | | 60000 TF@ 10 | 4700 |
| | Glucoside | Isostearate | 25 | | 36500 TF@ 20 | |
| | 30 | (and) | | | | |
| | | Caprylic/ | | | | |
| | | Capric | | | | |
| | | Glycerides | | | | |
| | | 15 | | | | |
| 71a | Decyl | Glyceryl | Zinc Oxide | | 126000 TF@10 | 11600 |
| | Glucoside | Stearate | 25 | | 68000 TF@20 | |
| | 47 | 12 | | | | |
| 72 | Decyl | Sorbitan | Zinc Oxide | | 110000 TF@ 10 | 8800 |
| | Glucoside | Oleate | 25 | | 66000 TF@ 20 | |
| | 28.13 | 18.75 | | | | |
| 73 | Decyl | Glyceryl | Homosalate | | 134000 TF@ 10 | 10100 |
| | Glucoside | Isostearate | 15 | | 83500 TF@ 20 | |
| | 34 | (and) | | | | |
| | | Caprylic/ | | | | |
| | | Capric | | | | |
| | | Glycerides | | | | |
| | | 17 | | | | |
| 74 | Decyl | Glyceryl | Homosalate | Cetyl/Lauryl/ | 170000 TF@ 10 | 15200 |
| | Glucoside | Isostearate | 15 | Myristyl | 94000 TF@ 20 | |
| | 30 | (and) | | Benzoate | | |
| | | Caprylic/ | | 10 | | |
| | | Capric | | | | |
| | | Glycerides | | | | |
| | | 15 | | | | |
| 75 | Decyl | Glyceryl | Octocrylene | | 255000 TF@ 10 | 23200 |
| | Glucoside | Isostearate | 10 | | 139000 TF@ 20 | |
| | 36 | (and) | | | | |
| | | Caprylic/ | | | | |
| | | Capric | | | | |
| | | Glycerides | | | | |
| | | 18 | | | | |
| 76 | Decyl | Glyceryl | Octocrylene | Cetyl/Lauryl/ | 124000 TF@ 10 | 10900 |
| | Glucoside | Isostearate | 10 | Myristyl | 69500 TF@ 20 | |
| | 30 | (and) | | Benzoate | | |
| | | Caprylic/ | | 15 | | |
| | | Capric | | | | |
| | | Glycerides | | | | |
| | | 15 | | | | |
| 77 | Decyl | Glyceryl | Avobenzone | | 340000 TF@ 10 | 40200 |
| | Glucoside | Isostearate | 3 | | 139000 TF@ 20 | |
| | 38.75 | (and) | | | | |
| | | Caprylic/ | | | | |
| | | Capric | | | | |
| | | Glycerides | | | | |
| | | 19.5 | | | | |
| 78 | Decyl | Glyceryl | Avobenzone | Cetyl/Lauryl/ | 176000 TF@ 10 | 15200 |
| | Glucoside | Isostearate | 3 | Myristyl | 100000 TF@ 20 | |
| | 30 | (and) | | Benzoate | | |
| | | Caprylic/ | | 22 | | |
| | | Capric | | | | |
| | | Glycerides | | | | |
| | | 15 | | | | |
| 79 | Decyl | Glyceryl | Octisalate | | 340000 TF@ 10 | 37200 |
| | Glucoside | Isostearate | 5 | | 154000 TF@ 20 | |
| | 38 | (and) | | | | |
| | | Caprylic/ | | | | |
| | | Capric | | | | |
| | | Glycerides | | | | |
| | | 19 | | | | |
| 80 | Decyl | Glyceryl | Octisalate | Cetyl/Lauryl/ | 155000 TF@ 10 | 13600 |
| | Glucoside | Isostearate | 5 | Myristyl | 87000 TF@ 20 | |
| | 30 | (and) | | Benzoate | | |
| | | Caprylic/ | | 20 | | |
| | | Capric | | | | |
| | | Glycerides | | | | |
| | | 15 | | | | |

### Polyglyceryl Examples

A series of formulations were prepared with various surfactants and polygylceryl ester structurants. The samples, preparation conditions, the BYV, and Stability are provided in Table 4. In Table 4 “Blend” indicates a mixture of Sodium Cocoyl Isethionate, Cocamidopropyl Hydroxysultaine, Lauryl Glucoside, Cocamidopropylamine Oxide and Caprylyl/Capryl Glucoside. In Table 4, BYV is the Brookfield Yield Value calculated as discussed above. In Table 4, Stability is defined as “A” indicating Stable wherein the dispersion did not separate over a period of about 5-6 weeks; “B” indicating Somewhat Stable wherein the dispersion did not separate for at least 1 day to no longer than about 5-6 weeks and “C” indicating Unstable where the dispersion was stable less than a day.

TABLE 4

| | Surfactant | | | |
|---|---|---|---|---|
| | Structurant | | | |
| | % Structurant/ | | Stability | |
| # | % Surfactant | BYV, dyn/cm² | A/B/C | Conditions |
| 81 | Cocamidopropyl | 470 | A | Structurant was |
| | Betaine | | | added to |
| | Polyglyceryl-3 | | | surfactant at |
| | Oleate | | | room |
| | 20/33.33 | | | temperature, |
| | | | | blended with |
| | | | | spatula, |
| | | | | thenheated to 50 |
| | | | | C. for 10 minutes. |
| | | | | Cooled to room |
| | | | | temperature. |
| 82 | Cocamidopropyl | 60 | B | Added water to |
| | Betaine | | | composition 81 |
| | Polyglyceryl-3 | | | at room |
| | Oleate | | | temperature, |
| | 10/16.67 | | | mixed with stir |
| | | | | bar for 10 |
| | | | | minutes. |
| 83 | Cocamidopropyl | 550 | C | Structurant was |
| | Betaine | | | added to |
| | Polyglyceryl-6 | | | surfactant at |
| | Oleate | | | room |
| | 20/33 | | | temperature, |
| | | | | blended with |
| | | | | spatula, |
| | | | | thenheated to 50 |
| | | | | C. for 10 minutes. |
| | | | | Cooled to room |
| | | | | temperature. |
| 84 | Cocamidopropyl | 0 | B | Added water to |
| | Betaine | | | composition 83 |
| | Polyglyceryl-6 | | | at room |
| | Oleate | | | temperature, |
| | 10/17 | | | mixed with stir |
| | | | | bar for 10 |
| | | | | minutes. |
| 85 | Cocamidopropyl | 0 | C | Structurant was |
| | Betaine | | | mixed with |
| | Polyglyceryl-10 | | | surfactant at |
| | Caprylate/Caprate | | | room |
| | 20/33.33 | | | temperature with |
| | | | | spatula, allowed |
| | | | | to sit to 30 |
| | | | | minutes to |
| | | | | thicken, stirring |
| | | | | every 10 min. |
| | | | | Then heated to |
| | | | | 50 C. for 10 |
| | | | | minutes. Cooled |
| | | | | to room |
| | | | | temperature. |
| 86 | Cocamidopropyl | 60 | B | Structurant was |
| | Betaine | | | mixed with |
| | Polyglyceryl-10 | | | surfactant at |
| | Polyhydroxystearate | | | room |
| | 20/33.33 | | | temperature with |
| | | | | spatula, allowed |
| | | | | to sit to 30 |
| | | | | minutes to |
| | | | | thicken, stirring |
| | | | | every 10 min. |
| | | | | Then heated to |
| | | | | 50 C. for 10 |
| | | | | minutes. Cooled |
| | | | | to room |
| | | | | temperature. |
| 87 | Sodium Lauroyl | 5350 | B | Structurant was |
| | Sarcosinate | | | mixed with |
| | Polyglyceryl-3 | | | surfactant at |
| | Oleate | | | room |
| | 20/33.33 | | | temperature, |
| | | | | blended with |
| | | | | spatula, |
| | | | | thenheated to 50 |
| | | | | C. for 10 minutes. |
| 88 | Sodium Lauroyl | 50 | B | Added water to |
| | Sarcosinate | | | composition 87 |
| | Polyglyceryl-3 | | | at room |
| | Oleate | | | temperature, |
| | 12/19.98 | | | mixed with stir |
| | | | | bar for 20 |
| | | | | minutes, then |
| | | | | heated to 50-70 C. |
| | | | | for 10 minutes. |
| 89 | Sodium Lauroyl | 3300 | A | Structurant was |
| | Sarcosinate | | | mixed with |
| | Polyglyceryl-4 | | | surfactant at |
| | Oleate | | | room |
| | 20/33.33 | | | temperature, |
| | | | | blended with |
| | | | | spatula, |
| | | | | thenheated to 50 |
| | | | | C. for 10 minutes. |
| 90 | Sodium Lauroyl | 0 | C | Structurant was |
| | Sarcosinate | | | mixed with |
| | Polyglyceryl-10 | | | surfactant at |
| | Caprylate/Caprate | | | room |
| | 20/33.33 | | | temperature, |
| | | | | blended with |
| | | | | spatula, |
| | | | | thenheated to 50 |
| | | | | C. for 10 minutes. |
| 91 | Sodium Lauroyl | 80 | B | Structurant was |
| | Sarcosinate | | | mixed with |
| | Polyglyceryl-10 | | | surfactant at |
| | Polyhydroxystearate | | | room |
| | 20/33.33 | | | temperature, |
| | | | | blended with |
| | | | | spatula, |
| | | | | thenheated to 50 |
| | | | | C. for 10 minutes. |
| 92 | Decyl Glucoside | 1480 | A | Structurant was |
| | and Sodium Lauroyl | | | mixed with |
| | Lactylate | | | surfactant at |
| | Polyglyceryl-3 | | | room |
| | Oleate | | | temperature, |
| | 36/33.33 | | | blended with |
| | | | | spatula, |
| | | | | thenheated to 50 |
| | | | | C. for 10 minutes. |
| 93 | Decyl Glucoside | 50 | C | Added water to |
| | and Sodium Lauroyl | | | composition 92 |
| | Lactylate | | | at room |
| | Polyglyceryl-3 | | | temperature, |
| | Oleate | | | mixed with stir |
| | 22/19.98 | | | bar for 20 |
| | | | | minutes. |
| 94 | Decyl Glucoside | 1620 | A | Structurant was |
| | and Sodium Lauroyl | | | mixed with |
| | Lactylate | | | surfactant at |
| | Polyglyceryl-3 | | | room |
| | Oleate | | | temperature, |
| | 36/33.33 | | | blended with |
| | | | | spatula, then |
| | | | | heated to 50 C. |
| | | | | for 10 minutes. |
| 95 | Decyl Glucoside | 30 | C | Added water to |
| | and Sodium Lauroyl | | | composition 94 |
| | Lactylate | | | at room |
| | Polyglyceryl-3 | | | temperature, |
| | Oleate | | | mixed with stir |
| | 22/19.98 | | | bar for 20 |
| | | | | minutes. |
| 96 | Decyl Glucoside | 600 | A | Structurant was |
| | and Sodium Lauroyl | | | mixed with |
| | Lactylate | | | surfactant at |
| | Polyglyceryl-4 | | | room |
| | Oleate | | | temperature, |
| | 36/33.33 | | | blended with |
| | | | | spatula, |
| | | | | thenheated to 50 |
| | | | | C. for 10 minutes. |
| 97 | Decyl Glucoside | 20 | C | Added water to |
| | and Sodium Lauroyl | | | composition 96 |
| | Lactylate | | | at room |
| | Polyglyceryl-4 | | | temperature, |
| | Oleate | | | mixed with stir |
| | 22/19.98 | | | bar for 20 |
| | | | | minutes. |
| 98 | Decyl glucoside and | 300 | C | Surfactant was |
| | Sodium Lauroyl | | | mixed with |
| | Lactylate | | | structurant at |
| | Polyglyceryl-6 | | | room |
| | Oleate | | | temperature, |
| | 36/33 | | | blended with |
| | | | | spatula, |
| | | | | thenheated to 50 |
| | | | | C. for 10 minutes. |
| 99 | Decyl glucoside and | 150 | C | Added water to |
| | Sodium Lauroyl | | | composition 98 |
| | Lactylate | | | at room |
| | Polyglyceryl-6 | | | temperature, |
| | Oleate | | | mixed with stir |
| | 22/20 | | | bar for 20 |
| | | | | minutes. |
| 100 | Decyl Glucoside | 0 | C | Structurant was |
| | and Sodium Lauroyl | | | mixed with |
| | Lactylate | | | surfactant at |
| | Polyglyceryl-10 | | | room |
| | Caprylate/Caprate | | | temperature, |
| | 36/33.33 | | | blended with |
| | | | | spatula, |
| | | | | thenheated to 50 |
| | | | | C. for 10 minutes. |
| 101 | Decyl Glucoside | 450 | B | Structurant was |
| | and Sodium Lauroyl | | | mixed with |
| | Lactylate | | | surfactant at |
| | Polyglyceryl-10 | | | room |
| | Polyhydroxystearate | | | temperature, |
| | 36/33.33 | | | blended with |
| | | | | spatula, |
| | | | | thenheated to 50 |
| | | | | C. for 10 minutes. |
| 102 | Decyl Glucoside | 0 | C | Added water to |
| | and Sodium Lauroyl | | | composition 101 |
| | Lactylate | | | at room |
| | Polyglyceryl-10 | | | temperature, |
| | Polyhydroxystearate | | | mixed with stir |
| | 22/19.98 | | | bar for 20 |
| | | | | minutes. |
| 103 | Behentrimonium | 11800 | A | Structurant was |
| | Chloride | | | added to a 20% |
| | Polyglyceryl-3 | | | solution of |
| | Oleate | | | surfactant at |
| | 13.90/14 | | | room |
| | | | | temperature and |
| | | | | mixed with |
| | | | | spatula. |
| 104 | Behentrimonium | 9800 | A | Structurant was |
| | Chloride | | | added to a 20% |
| | Polyglyceryl-3 | | | solution of |
| | Oleate | | | surfactant at |
| | 13.90/14 | | | room |
| | | | | temperature and |
| | | | | mixed with |
| | | | | spatula. |
| 105 | Behentrimonium | 11600 | A | Structurant was |
| | Chloride | | | added to a 20% |
| | Polyglyceryl-4 | | | solution of |
| | Oleate | | | surfactant at |
| | 13.90/14 | | | room |
| | | | | temperature and |
| | | | | mixed with |
| | | | | spatula. |
| 106 | Behentrimonium | 8100 | A | Structurant was |
| | Chloride | | | added to a 20% |
| | Polyglyceryl-4 | | | solution of |
| | Oleate | | | surfactant at |
| | 13.90/14 | | | room |
| | | | | temperature and |
| | | | | mixed with |
| | | | | spatula. |
| 107 | Behetrimonium | 10750 | A | Structurant was |
| | Chloride | | | added to a 20% |
| | Polyglyceryl-6 | | | solution of |
| | Oleate | | | surfactant at |
| | 14/14 | | | room |
| | | | | temperature and |
| | | | | mixed with |
| | | | | spatula. |
| 108 | Behentrimonium | 1000 | A | Structurant was |
| | Chloride | | | added to a 20% |
| | Polyglyceryl-10 | | | solution of |
| | Caprylate/Caprate | | | surfactant at |
| | 13.90/14 | | | room |
| | | | | temperature and |
| | | | | mixed with |
| | | | | spatula. |
| 109 | Behentrimonium | 560 | A | Structurant was |
| | Chloride | | | added to a 20% |
| | Polyglyceryl-10 | | | solution of |
| | Polyhydroxystearate | | | surfactant at |
| | 13.90/14 | | | room |
| | | | | temperature and |
| | | | | mixed with |
| | | | | spatula. |
| 110 | Disodium Laureth | 1150 | A | Added |
| | Sulfosuccinate | | | structurant to |
| | Polyglyceryl-3 | | | surfactant at |
| | Oleate | | | room |
| | 24.64/23 | | | temperature, |
| | | | | mixed with |
| | | | | spatula. |
| 111 | Disodium Laureth | 800 | A | Added water to |
| | Sulfosuccinate | | | composition 110 |
| | Polyglyceryl-3 | | | at room |
| | Oleate | | | temperature, |
| | 21.44/20 | | | mixed with |
| | | | | spatula. |
| 112 | Disodium Laureth | 1500 | A | Added |
| | Sulfosuccinate | | | structurant to |
| | Polyglyceryl-3 | | | surfactant at |
| | Oleate | | | room |
| | 24.64/23 | | | temperature, |
| | | | | mixed with |
| | | | | spatula. |
| 113 | Disodium Laureth | 1150 | A | Added water to |
| | Sulfosuccinate | | | composition 112 |
| | Polyglyceryl-3 | | | at room |
| | Oleate | | | temperature, |
| | 21.44/20 | | | mixed with |
| | | | | spatula. |
| 114 | Disodium Laureth | 1000 | A | Added |
| | Sulfosuccinate | | | structurant to |
| | Polyglyceryl-4 | | | surfactant at |
| | Oleate | | | room |
| | 24.64/23 | | | temperature, |
| | | | | mixed with prop |
| | | | | blade. |
| 115 | Disodium Laureth | 2400 | B | Added water to |
| | Sulfosuccinate | | | composition 114 |
| | Polyglyceryl-4 | | | at room |
| | Oleate | | | temperature, |
| | 21.44/20 | | | mixed with prop |
| | | | | blade. |
| 116 | Disodium Laureth | 2800 | B | Added |
| | Sulfosuccinate | | | structurant to |
| | Polyglyceryl-6 | | | surfactant at |
| | Oleate | | | room |
| | 25/23 | | | temperature, |
| | | | | mixed with |
| | | | | spatula. |
| 117 | Disodium Laureth | 200 | B | Added water to |
| | Sulfosuccinate | | | composition 116 |
| | Polyglyceryl-6 | | | at room |
| | Oleate | | | temperature, |
| | 21/20 | | | mixed with |
| | | | | spatula. |
| 118 | Disodium Laureth | 0 | C | Added |
| | Sulfosuccinate | | | structurant to |
| | Polyglyceryl-10 | | | surfactant at |
| | Caprylate/Caprate | | | room |
| | 24.64/23 | | | temperature, |
| | | | | mixed with |
| | | | | spatula. |
| 119 | Disodium Laureth | 0 | C | Added water to |
| | Sulfosuccinate | | | composition 118 |
| | Polyglyceryl-10 | | | at room |
| | Caprylate/Caprate | | | temperature, |
| | 21.44/20 | | | mixed with |
| | | | | spatula. |
| 120 | Disodium Laureth | 50 | B | Added |
| | Sulfosuccinate | | | structurant to |
| | Polyglyceryl-10 | | | surfactant at |
| | Polyhydroxystearate | | | room |
| | 24.64/23 | | | temperature, |
| | | | | mixed with |
| | | | | spatula. |
| 121 | Blend | 2200 | A | Surfactant was |
| | Polyglyceryl-3 | | | mixed with |
| | Oleate | | | structurant and |
| | 23/23 | | | water at room |
| | | | | temperature, |
| | | | | blended with |
| | | | | spatula after |
| | | | | each addition, |
| | | | | then heated to 50 |
| | | | | C. for 10 minutes. |
| 122 | Blend | 1800 | A | Water was added |
| | Polyglyceryl-3 | | | to composition |
| | Oleate | | | 121 at room |
| | 20/20 | | | temperature and |
| | | | | mixed with |
| | | | | spatula. |
| 123 | Blend | 2400 | A | Surfactant was |
| | Polyglyceryl-3 | | | mixed with |
| | Oleate | | | structurant and |
| | 23/23 | | | water at room |
| | | | | temperature, |
| | | | | blended with |
| | | | | spatula after |
| | | | | each addition, |
| | | | | then heated to 50 |
| | | | | C. for 10 minutes. |
| 124 | Blend | 2100 | A | Water was added |
| | Polyglyceryl-3 | | | to composition |
| | Oleate | | | 123 at room |
| | 20/20 | | | temperature and |
| | | | | mixed with |
| | | | | spatula. |
| 125 | Blend | 1000 | A | Surfactant was |
| | Polyglyceryl-4 | | | mixed with |
| | Oleate | | | structurant and |
| | 23/23 | | | water at room |
| | | | | temperature, |
| | | | | blended with |
| | | | | spatula after |
| | | | | each addition, |
| | | | | then heated to 50 |
| | | | | C. for 10 minutes. |
| 126 | Blend | 1050 | A | Water was added |
| | Polyglyceryl-4 | | | to composition |
| | Oleate | | | 125 at room |
| | 20/20 | | | temperature and |
| | | | | mixed with |
| | | | | spatula. |
| 127 | Blend | 900 | A | Surfactant was |
| | Polyglyceryl-4 | | | mixed with |
| | Oleate | | | structurant and |
| | 23/23 | | | water at room |
| | | | | temperature, |
| | | | | blended with |
| | | | | spatula after |
| | | | | each addition, |
| | | | | then heated to 50 |
| | | | | C. for 10 minutes. |
| 128 | Blend | 850 | A | Water was added |
| | Polyglyceryl-4 | | | to composition |
| | Oleate | | | 127 at room |
| | 20/20 | | | temperature and |
| | | | | mixed with |
| | | | | spatula. |
| 129 | Blend | 150 | B | Surfactant was |
| | Polyglyceryl-6 | | | mixed with |
| | Oleate | | | structurant and |
| | 23/23 | | | water at room |
| | | | | temperature, |
| | | | | blended with |
| | | | | spatula after |
| | | | | each addition, |
| | | | | then heated to 50 |
| | | | | C. for 10 minutes. |
| 130 | Blend | 500 | B | Water was added |
| | Polyglyceryl-6 | | | to composition |
| | Oleate | | | 129 at room |
| | 20/20 | | | temperature and |
| | | | | mixed with |
| | | | | spatula. |
| 131 | Blend | 250 | C | Surfactant was |
| | Polyglyceryl-6 | | | mixed with |
| | Oleate | | | structurant and |
| | 20/32 | | | water at room |
| | | | | temperature, |
| | | | | blended with |
| | | | | spatula after |
| | | | | each addition, |
| | | | | then heated to 50 |
| | | | | C. for 10 minutes. |
| 132 | Blend | 850 | B | Equal parts of |
| | Polyglyceryl-6 | | | 131 and water |
| | Oleate | | | were combined |
| | 10/16 | | | at room |
| | | | | temperature and |
| | | | | mixed with |
| | | | | spatula. |
| 133 | Blend | 100 | C | Surfactant was |
| | Polyglyceryl-10 | | | mixed with |
| | Caprylate/Caprate | | | structurant and |
| | 23/23 | | | water at room |
| | | | | temperature, |
| | | | | blended with |
| | | | | spatula after |
| | | | | each addition, |
| | | | | then heated to 50 |
| | | | | C. for 10 minutes. |
| 134 | Blend | 0 | C | Water was added |
| | Polyglyceryl-10 | | | to composition |
| | Caprylate/Caprate | | | 133 at room |
| | 20/20 | | | temperature and |
| | | | | mixed with |
| | | | | spatula. |
| 135 | Blend | 5550 | A | Surfactant was |
| | Polyglyceryl-3 | | | mixed with |
| | Oleate | | | structurant and |
| | 20/32 | | | water at room |
| | | | | temperature, |
| | | | | blended with |
| | | | | spatula after |
| | | | | each addition, |
| | | | | then heated to 50 |
| | | | | C. for 10 minutes. |
| 136 | Blend | 1050 | A | Equal parts of |
| | Polyglyceryl-3 | | | 135 and water |
| | Oleate | | | were combined |
| | 10/16 | | | at room |
| | | | | temperature and |
| | | | | mixed with |
| | | | | spatula. |
| 137 | Blend | 7300 | A | Surfactant was |
| | Polyglyceryl-3 | | | mixed with |
| | Oleate | | | structurant and |
| | 20/32 | | | water at room |
| | | | | temperature, |
| | | | | blended with |
| | | | | spatula after |
| | | | | each addition, |
| | | | | then heated to 50 |
| | | | | C. for 10 minutes. |
| 138 | Blend | 900 | A | Equal parts of |
| | Polyglyceryl-3 | | | 137 and water |
| | Oleate | | | were combined |
| | 10/16 | | | at room |
| | | | | temperature and |
| | | | | mixed with |
| | | | | spatula. |
| 139 | Blend | 5000 | A | Surfactant was |
| | Polyglyceryl-4 | | | mixed with |
| | Oleate | | | structurant and |
| | 20/32 | | | water at room |
| | | | | temperature, |
| | | | | blended with |
| | | | | spatula after |
| | | | | each addition, |
| | | | | then heated to 50 |
| | | | | C. for 10 minutes. |
| 140 | Blend | 450 | A | Equal parts of |
| | Polyglyceryl-4 | | | composition 139 |
| | Oleate | | | and water were |
| | 10/16 | | | combined at |
| | | | | room |
| | | | | temperature and |
| | | | | mixed with |
| | | | | spatula. |
| 141 | Blend | 4800 | A | Surfactant was |
| | Polyglyceryl-4 | | | mixed with |
| | Oleate | | | structurant and |
| | 20/32 | | | water at room |
| | | | | temperature, |
| | | | | blended with |
| | | | | spatula after |
| | | | | each addition, |
| | | | | then heated to 50 |
| | | | | C. for 10 minutes. |
| 142 | Blend | 350 | A | Equal parts of |
| | Polyglyceryl-4 | | | composition 141 |
| | Oleate | | | and water were |
| | 10/16 | | | combined at |
| | | | | room |
| | | | | temperature and |
| | | | | mixed with |
| | | | | spatula. |
| 143 | Blend | 100 | C | Surfactant was |
| | Polyglyceryl-10 | | | mixed with |
| | Caprylate/Caprate | | | structurant and |
| | 20/32 | | | water at room |
| | | | | temperature, |
| | | | | blended with |
| | | | | spatula after |
| | | | | each addition, |
| | | | | then heated to 50 |
| | | | | C. for 10 minutes. |
| 144 | Blend | 0 | C | Equal parts of |
| | Polyglyceryl-10 | | | composition 143 |
| | Caprylate/Caprate | | | and water were |
| | 10/16 | | | combined at |
| | | | | room |
| | | | | temperature and |
| | | | | mixed with |
| | | | | spatula. |
| 145 | Decyl Glucoside | 150 | C | Added |
| | Polyglyceryl-3 | | | structurant to |
| | Oleate | | | surfactant at |
| | 37/23 | | | room |
| | | | | temperature, |
| | | | | mixed with |
| | | | | spatula. |
| 146 | Decyl Glucoside | 50 | C | Added water to |
| | Polyglyceryl-3 | | | composition 145 |
| | Oleate | | | at room |
| | 24.95/17.5 | | | temperature, |
| | | | | mixed with |
| | | | | spatula. |
| 147 | Decyl Glucoside | 0 | C | Added |
| | Polyglyceryl-4 | | | structurant to |
| | Oleate | | | surfactant at |
| | 37/26 | | | room |
| | | | | temperature, |
| | | | | mixed with |
| | | | | spatula. |
| 148 | Decyl Glucoside | 0 | C | Added water to |
| | Polyglyceryl-4 | | | composition 147 |
| | Oleate | | | at room |
| | 24.95/17.5 | | | temperature, |
| | | | | mixed with |
| | | | | spatula. |
| 149 | Decyl Glucoside | 200 | B | Added |
| | Polyglyceryl-2 | | | structurant to |
| | Oleate | | | surfactant at |
| | 37/26 | | | room |
| | | | | temperature, |
| | | | | mixed with |
| | | | | spatula. |
| 150 | Decyl Glucoside | 200 | A | Added water to |
| | Polyglyceryl-2 | | | composition 149 |
| | Oleate | | | at room |
| | | | | temperature, |
| | | | | mixed with |
| | | | | spatula. |
| 151 | Decyl Glucoside | 100 | C | Added |
| | Polyglyceryl-3 | | | structurant to |
| | Dioleate | | | surfactant at |
| | 26/37 | | | room |
| | | | | temperature, |
| | | | | mixed with |
| | | | | spatula. |
| 152 | Decyl Glucoside | 100 | C | Added water to |
| | Polyglyceryl-3 | | | composition 151 |
| | Dioleate | | | at room |
| | 24.95/17.5 | | | temperature, |
| | | | | mixed with |
| | | | | spatula. |
| 153 | Decyl Glucoside | 1050 | C | Added |
| | Polyglyceryl-2 | | | structurant to |
| | Oleate | | | surfactant at |
| | 33/33 | | | room |
| | | | | temperature, |
| | | | | mixed with |
| | | | | spatula. |
| 154 | Decyl Glucoside | 550 | C | Added water to |
| | Polyglyceryl-2 | | | composition 153 |
| | Oleate | | | at room |
| | 22/22 | | | temperature, |
| | | | | mixed with |
| | | | | spatula. |
| 155 | Decyl Glucoside | 9800 | A | Added |
| | Polyglyceryl-3 | | | structurant to |
| | Dioleate | | | surfactant at |
| | 33/33 | | | room |
| | | | | temperature, |
| | | | | mixed with |
| | | | | spatula. |
| 156 | Decyl Glucoside | 2000 | A | Added water to |
| | Polyglyceryl-3 | | | composition 155 |
| | Dioleate | | | at room |
| | 22/22 | | | temperature, |
| | | | | mixed with |
| | | | | spatula. |
| 157 | Decyl Glucoside | 1450 | A | Added |
| | Polyglyceryl-3 | | | structurant to |
| | Dioleate | | | surfactant at |
| | 30/41 | | | room |
| | | | | temperature, |
| | | | | mixed with |
| | | | | spatula. |
| 158 | Decyl Glucoside | 3050 | A | Added water to |
| | Polyglyceryl-3 | | | composition 157 |
| | Dioleate | | | at room |
| | 20/27 | | | temperature, |
| | | | | mixed with |
| | | | | spatula. |
| 159 | Decyl Glucoside | 950 | B | Added |
| | Polyglyceryl-2 | | | structurant to |
| | Oleate | | | surfactant at |
| | 23/54 | | | room |
| | | | | temperature, |
| | | | | mixed with |
| | | | | spatula. |
| 160 | Decyl Glucoside | 1650 | A | Added water to |
| | Polyglyceryl-2 | | | composition 159 |
| | Oleate | | | at room |
| | 15/36 | | | temperature, |
| | | | | mixed with |
| | | | | spatula. |
| 161 | Decyl glucoside | 100 | B | Added |
| | Polyglyceryl-6 | | | structurant to |
| | Oleate | | | surfactant at |
| | 37/26 | | | room |
| | | | | temperature, |
| | | | | mixed with |
| | | | | spatula. |

A Brookfield Yield Value (BYV) was observed for all surfactants tested-anionic, cationic, nonionic, & amphoteric using polyglyceryl esters. This BYV indicated a Non-Newtonian pseudoplastic rheology was observed for all surfactants tested including anionic, cationic, nonionic, and amphoteric surfactants using the polyglyceryl esters.

Total amount of structurant and surfactant is important to the rheology. The optimum structurant range was 14-54% and the optimum surfactant range was 10-37% with an optimum structurant/surfactant range was 0.7-2.3.

It was determined that heat is not necessarily needed to obtain the Rheology and post-adding water to the structurant/surfactant blend may or may not affect the Rheology.

HLB of the Structurant was important to the Rheology with the optimal being about no more than 8. The one exception was entry 105 with Behentrimonium Chloride since Polyglyceryl-10 Caprylate/Caprate has an HLB of 17.

The BYV range observed was 50-11,800 dyn/cm².

The invention has been described with reference to the preferred embodiments without limit thereto. Additional embodiments and improvements may be realized which are not specifically set forth herein but which are within the scope of the invention as more specifically set forth in the claims appended hereto.

Claimed is:

1. A colloidal dispersion having a Brookfield Yield Value of at least 50 dyn/cm² comprising:
a structurant wherein said structurant is a glyceryl ester or glyceryl ether; and
a sulfate-free surfactant.

2. The colloidal dispersion of claim 1 wherein said structurant has an HLB of at least 4 to no more than 18.

3. The colloidal dispersion of claim 1 wherein said colloidal dispersion has an HLB of at least 4 to no more than 12.

4. The colloidal dispersion of claim 1 wherein said colloidal dispersion has an HLB of at least 4 to no more than 8.

5. The colloidal dispersion of claim 1 wherein said colloidal dispersion has a Brookfield Viscosity of no more than 1,000,000 cps measured at a spindle speed of 10 rpm.

6. The colloidal dispersion of claim 1 further comprising a solvent.

7. The colloidal dispersion of claim 6 wherein said solvent is water.

8. The colloidal dispersion of claim 6 comprising 4-60 wt % said structurant, 6-45 wt % said surfactant and up to 90 wt % solvent.

9. The colloidal dispersion of claim 1 wherein said structurant is a polyglyceryl ether.

10. The colloidal dispersion of claim 1 wherein said structurant is a polyglyceryl ester.

11. The colloidal dispersion of claim 10 wherein said structurant is selected from the group consisting of polyglyceryl-2 oleate, polyglyceryl-3 oleate, polyglyceryl-4 oleate, polyglyceryl-10 caprylate/caprate, polyglyceryl-6 oleate, polyglyceryl-6 caprylate/caprate, polyglyceryl-10 polyhydroxystearate and polyglyceryl-3 dioleate.

12. The colloidal dispersion of claim 1 comprising less than 2 wt % sulfate-based surfactant.

13. The colloidal dispersion of claim 12 comprising less than 1 wt % sulfate-based surfactant.

14. The colloidal dispersion of claim 1 wherein said sulfate-free surfactant is selected from the group consisting polyalkylene glycol adducts of alkyl C8-C24 phenols; polyalkylene glycol adducts of C8-C30 alcohols or C8-C30 glycosides, linear or branched, saturated or unsaturated; polyalkylene glycol adducts of C8-C30, linear or branched, saturated or unsaturated fatty acid amides; C8-C30 linear or branched, saturated or unsaturated esters of sorbitol, which are preferably polyalkylene glycol adducts; fatty acid esters of sucrose; C8-C30 alkyl polyglycosides; C8-C30 alkenyl polyglycosides, which are optionally polyalkylene glycol adducts with from 0 to 10 polyalkylene glycol units and comprising from 1 to 15 glucose units; polyalkylene glycol adducts of saturated or unsaturated vegetable oils; derivatives of N-alkyl (C8-C30) glucamine; derivatives of N-acyl C8-C30 methylglucamine; aldobionamides; amine oxides; polyalkylene glycol adducts of silicones; polyglycerin adducts of fatty acids and alcohols including lauric acid or lauryl alcohol containing 1-20 moles of glycerin; polyglycerin adducts of oleic acid or oleyl alcohol containing 1-20 moles of glycerin; polyglycerin adducts of cetearic acid or cetearyl alcohol containing 1-20 moles of glycerin; polyglycerin adducts of octadecanic acid or octadecanol containing 1-20 moles of glycerin.

15. The colloidal dispersion of claim 1 wherein said sulfate-free surfactant is a non-ionic surfactant selected from the group consisting of decyl glucoside, lauryl glucoside, caprylic/capric glucoside and coco glucoside.

16. The colloidal dispersion of claim 1 wherein said cleaning system further comprises a surfactant selected from the group consisting of a cationic surfactant, an anionic surfactant and an amphoteric surfactant.

17. The colloidal dispersion of claim 16 wherein said surfactant is selected from the group consisting of glutamates, taurates, alkanoyl isethionates, alkyl succinates, alkyl sulphosuccinates, N-alkoyl sarcosinates, alkyl phosphates, alkyl ether phosphates, alkyl ether carboxylates, alpha-olefin sulphonates wherein alkyl and acyl groups are preferably 8 to 18 carbons which may be unsaturated. Particularly preferred alpha-olefin sulphonates include the sodium, magnesium, ammonium and mono-, di- and triethanolamine salts thereof. Particularly preferred anionic surfactants include sodium oleyl succinate, ammonium lauryl sulphosuccinate, disodium laureth sulfosuccinate, sodium dodecylbenzene sulphonate, triethanolamine dodecylbenzene sulphonate, sodium cocoyl isethionate, sodium lauroyl isethionate, sodium N-lauryl sarcosinate, sodium lauroyl lactylate, sodium lauroyl glutamate, sodium methyl cocoyl taurate, sodium lauroyl methyl taurate, sodium lauriminodipropionate and mixtures thereof.

18. The colloidal dispersion of claim 16 wherein said surfactant is selected from the group consisting of quaternized polysaccharides, alkyl polysaccharides, alkoxylated amines, alkoxylated ether amines, phospholipids, phospholipid derivatives and mixtures thereof.

19. The colloidal dispersion of claim 16 wherein said surfactant is selected from the group consisting of alkanoyl isethionates, alkyl succinates, alkyl sulphosuccinates, N-alkoyl sarcosinates, alkyl phosphates, alkyl ether phosphates, alkyl ether carboxylates, alpha-olefin sulphonates wherein alkyl and acyl groups comprise 8 to 18 carbons and may be unsaturated.

20. The colloidal dispersion of claim 16 wherein said surfactant is selected from the group consisting of sodium, magnesium, ammonium and mono-, di- and triethanolamine salts of alpha-olefin sulphonates.

21. The colloidal dispersion of claim 16 wherein said surfactant is selected from the group consisting of sodium oleyl succinate, ammonium lauryl sulphosuccinate, disodium laureth sulfosuccinate, sodium dodecylbenzene sulphonate, triethanolamine dodecylbenzene sulphonate, sodium cocoyl isethionate, sodium lauroyl isethionate, sodium N-lauryl sarcosinate, sodium lauroyl lactylate and sodium lauriminodipropionate.

22. The colloidal dispersion of claim 21 wherein said surfactant is sodium lauriminodipropionate.

23. The colloidal dispersion of claim 16 wherein said surfactant is selected from the group consisting of cocamidopropylhydroxtsultaine, cocamidopropyl betaine, cocobetaine, sodium salt of diethylaminopropyl laurylaminosuccinamate or mixtures thereof.

24. The colloidal dispersion of claim 16 wherein said second surfactant is selected from the group consisting of disodium laureth sulfosuccinate, decyl glucoside sodium lauroyl lactylate and sodium lauriminodipropionate.

25. A personal care product comprising the colloidal dispersion of claim 1.

26. The personal care product of claim 25 selected from the group consisting of soap, shampoo, body-wash, make-up remover.

27. The personal care product of claim 25 comprising a pigment.

28. The personal care product of claim 25 selected from the group consisting of mascara, eye-liner, lipstick and sunscreen.

29. A cleaning system comprising:
a colloidal dispersion comprising:
a structurant wherein said structurant is a glyceryl ester or glyceryl ether; and
a sulfate-free surfactant; and
a cleaning agent.

30. The cleaning system of claim 29 wherein said structurant is a polyglyceryl ether.

31. The cleaning system of claim 29 wherein said structurant is a polyglyceryl ester.

32. The cleaning system of claim 31 wherein said structurant is selected from the group consisting of polyglyceryl-2 oleate, polyglyceryl-3 oleate, polyglyceryl-4 oleate, polyglyceryl-10 caprylate/caprate, polyglyceryl-6 oleate, polyglyceryl-6 caprylate/caprate, polyglyceryl-10 polyhydroxystearate and polyglyceryl-3 dioleate.

33. The cleaning system of claim 29 wherein said structurant has an HLB of at least 4 to no more than 18.

34. The cleaning system of claim 29 wherein said colloidal dispersion has an HLB of at least 4 to no more than 12.

35. The cleaning system of claim 29 wherein said colloidal dispersion has a Brookfiel Yield Value of at least 50 dyn/cm².

36. The cleaning system of claim 29 wherein said colloidal dispersion has a Brookfield Viscosity of no more than 1,000,000 cps measured at a spindle speed of 10 rpm.

37. The cleaning system of claim 29 comprising 10-30 wt % said structurant, 20-40 wt % said sulfate-free surfactant and 30-70 wt % said cleaning agent.

38. The cleaning system of claim 29 comprising 10-95 wt % said colloidal dispersion and 5-90 wt % said cleaning agent.

39. The cleaning system of claim 29 wherein said cleaning agent is selected from the group consisting of esters, propylene carbonates, terpenes, N-methylpyrrolidone, acetates, and ethers.

40. The cleaning system of claim 29 wherein said cleaning agent is selected from the group consisting of alkyl esters with up to 5 carbons which may be substituted; cyclic terpenes; dipropylene glycol methyl ether acetate, dipropylene glycol monomethyl ether acetate, diethylene glycol monoethyl ether acetate, ethyl acetate, n-propyl acetate, n-butyl acetate, isobutyl acetate, dipropylene glycol methyl ether, and diethylene glycol ethyl ether.

41. The cleaning system of claim 29 wherein said cleaning agent is selected from the group consisting of methyl ester, ethyl ester, 3-ethoxypropionic ethyl ester, ethyl lactate, soy methyl ester, triglyceride methyl ester, C₁₋₄ alkyl ester of a C₆₋₂₂ saturated or unsaturated carboxylic acid, and limonene.

42. The cleaning system of claim 29 further comprising a solvent.

43. The cleaning system of claim 42 wherein said solvent is water.

44. The cleaning system of claim 29 comprising 4-50 wt % said structurant, 6-45 wt % said sulfate-free surfactant and up to 90 wt % said solvent.

45. The cleaning system of claim 29 comprising less than 2 wt % sulfate-based surfactant.

46. The cleaning system of claim 45 comprising less than 1 wt % sulfate-based surfactant.

47. The cleaning system of claim 29 wherein said sulfate-free surfactant is a non-ionic surfactant selected from the group consisting polyalkylene glycol adducts of alkyl C8-C24 phenols; polyalkylene glycol adducts of C8-C30 alcohols or C8-C30 glycosides, linear or branched, saturated or unsaturated; polyalkylene glycol adducts of C8-C30, linear or branched, saturated or unsaturated fatty acid amides; C8-C30 linear or branched, saturated or unsaturated esters of sorbitol, which are preferably polyalkylene glycol adducts; fatty acid esters of sucrose; C8-C30 alkyl polyglycosides; C8-C30 alkenyl polyglycosides, which are optionally polyalkylene glycol adducts with from 0 to 10 polyalkylene glycol units and comprising from 1 to 15 glucose units; polyalkylene glycol adducts of saturated or unsaturated vegetable oils; derivatives of N-alkyl (C8-C30) glucamine; derivatives of N-acyl C8-C30 methylglucamine; aldobionamides; amine oxides; polyalkylene glycol adducts of silicones; polyglycerin adducts of fatty acids and alcohols including lauric acid or lauryl alcohol containing 1-20 moles of glycerin; polyglycerin adducts of oleic acid or oleyl alcohol containing 1-20 moles of glycerin; polyglycerin adducts of cetearic acid or cetearyl alcohol containing 1-20 moles of glycerin; polyglycerin adducts of octadecanic acid or octadecanol containing 1-20 moles of glycerin.

48. The cleaning system of claim 29 wherein said sulfate-free surfactant is selected from the group consisting of decyl glucoside, lauryl glucoside, caprylic/capric glucoside and coco glucoside.

49. The cleaning system of claim 29 further comprising a second surfactant selected from the group consisting of a cationic surfactant, an anionic surfactant, a nonionic surfactant and an amphoteric surfactant.

50. The cleaning system of claim 49 wherein said second surfactant is selected from the group consisting of glutamates, taurates, alkanoyl isethionates, alkyl succinates, alkyl sulphosuccinates, N-alkoyl sarcosinates, alkyl phosphates, alkyl ether phosphates, alkyl ether carboxylates, alpha-olefin sulphonates wherein alkyl and acyl groups are preferably 8 to 18 carbons which may be unsaturated. Particularly preferred alpha-olefin sulphonates include the sodium, magnesium, ammonium and mono-, di- and triethanolamine salts thereof. Particularly preferred anionic surfactants include sodium oleyl succinate, ammonium lauryl sulphosuccinate, disodium laureth sulfosuccinate, sodium dodecylbenzene sulphonate, triethanolamine dodecylbenzene sulphonate, sodium cocoyl isethionate, sodium lauroyl isethionate, sodium N-lauryl sarcosinate, sodium lauroyl lactylate, sodium lauroyl glutamate, sodium methyl cocoyl taurate, sodium lauroyl methyl taurate, sodium lauriminodipropionate and mixtures thereof.

51. The cleaning system of claim 49 wherein said second surfactant is selected from the group consisting of quaternized polysaccharides, alkyl polysaccharides, alkoxylated amines, alkoxylated ether amines, phospholipids, phospholipid derivatives and mixtures thereof.

52. The cleaning system of claim 49 wherein said second surfactant is selected from the group consisting of alkanoyl isethionates, alkyl succinates, alkyl sulphosuccinates, N-alkoyl sarcosinates, alkyl phosphates, alkyl ether phosphates, alkyl ether carboxylates, alpha-olefin sulphonates wherein alkyl and acyl groups comprise 8 to 18 carbons and may be unsaturated.

53. The cleaning system of claim 49 wherein said second surfactant is selected from the group consisting of sodium, magnesium, ammonium and mono-, di- and triethanolamine salts of alpha-olefin sulphonates.

54. The cleaning system of claim 49 wherein said second surfactant is selected from the group consisting of sodium oleyl succinate, ammonium lauryl sulphosuccinate, disodium laureth sulfosuccinate, sodium dodecylbenzene sulphonate, triethanolamine dodecylbenzene sulphonate, sodium cocoyl isethionate, sodium lauroyl isethionate, sodium N-lauryl sarcosinate, sodium lauroyl lactylate and sodium lauriminodipropionate.

55. The cleaning system of claim 54 wherein said second surfactant is sodium lauriminodipropionate.

56. The cleaning system of claim 49 wherein said second surfactant is selected from the group consisting of cocamidopropylhtdroxtsultaine, cocamidopropyl betaine, cocobetaine, sodium salt of diethylaminopropyl laurylaminosuccinamate or mixtures thereof.

57. The cleaning system of claim 49 wherein said second surfactant is selected from the group consisting of disodium laureth sulfosuccinate, decyl glucoside sodium lauroyl lactylate and sodium lauriminodipropionate.
